(19) United States
(12) Patent Application Publication
(10) Pub. No.: US 2024/0263256 A1
(43) Pub. Date: Aug. 8, 2024

(54) CRISPR-Cas13 System, Kit and Method for Detecting SARS-CoV-2

(71) Applicant: Rafael Biotechnology Company Limited, Hong Kong (CN)

(72) Inventor: Siu Pang Ng, Hong Kong (CN)

(21) Appl. No.: 18/197,987

(22) Filed: May 16, 2023

(30) Foreign Application Priority Data
Feb. 6, 2023 (CN) 202310107029.9

(51) Int. Cl. C12Q 1/70; C12N 9/22; C12N 15/11

(52) U.S. Cl. CPC C12Q 1/701; C12N 9/22; C12N 15/11; C12N 2310/20

(57) ABSTRACT

Provided herein is a CRISPR-Cas13 system, a kit and a method for detecting SARS-COV-2. The CRISPR-Cas13a system includes a Cas13a protein and crRNA, or a complex formed by Cas13a protein and crRNA. The crRNA includes a first guide RNA and a second guide RNA, the first guide RNA and the second guide RNA having at least one sequence selected from SEQ ID NOs: 1 to 33, respectively.

### CROSS-REFERENCE TO RELATED APPLICATION

This application claims priority to Chinese Patent Application No. 202310107029.9 filed Feb. 6, 2023, the disclosure of which is hereby incorporated by reference in its entirety.

The Sequence Listing associated with this application is filed in electronic format via Patent Center and is hereby incorporated by reference into the specification in its entirety. The name of the file containing the Sequence Listing is 2304116.xml. The size of the file is 119,060 bytes, and the file was created on Apr. 28, 2023.

### TECHNICAL FIELD

The present disclosure is in the technical field of molecular diagnosis and specifically relates to a CRISPR-Cas13 system, a kit and a method for detecting SARS-COV-2.

### BACKGROUND

Severe Acute Respiratory Syndrome Coronavirus-2 (SARS-COV-2) is a single-stranded positive-sense RNA virus, and its functional coding genes include Open Reading Frame lab (ORF1ab), spike protein (S), envelope protein (E), membrane (M) and nucleocapsid (N) genes. SARS-COV-2 infection can result in Corona Virus Disease 2019 (COVID-19), patients may develop flu-like symptoms such as fever, cough, chest tightness and fatigue, etc., and in severe cases, dyspnea, acute respiratory distress syndrome and even death may occur. The source of infection for COVID-19 is a SARS-COV-2 infected patient, and the SARS-COV-2 are rapidly spread among the population through various routes such as respiratory droplets, direct contact with the SARS-COV-2 contamination and fecal-oral route, and the like. All of the people are susceptible to the SARS-COV-2. Currently, the detection and diagnosis methods of the SARS-COV-2 include nucleic acid test, immunological test and virus isolation and culture, among which nucleic acid test is the most recognized detection method.

The SARS-COV-2 evolves continuously with mutation in almost every part of its gene, as the result, the false negative rate of existing nucleic acid amplification test (NAT) is up to 30%. Currently, the best state-of-the-art nucleic acid test is based on quantitative reverse transcription-polymerase chain reaction (qRT-PCR), there are a number of limitations in dealing with mutations. First of all, the target RNA viral sequence is rather short which is usually less than 100 nucleotide bases in length. Secondly, only one sequence is selected from either ORF1ab or N or RdRp or E or S genes as target, and two genes are selected in combination. Thirdly, once the target sequence is selected, the complimentary forward and reverse primers as well as the fluorophore probe are synthesized based on the selected sequence. Although being extremely sensitive to the sequence, sensitivity of the qRT-PCR assay to mutation is subjected to question. Therefore, WHO suggests to routinely test all specimens with two different primer and probe sets that target different genomic regions to reduce the risk of false negative results.

In April 2017, some researchers combined the non-specific cleavage activity of Leptotrichia wadei Cas13a protein (LwCas13a) with recombinase polymerase amplification (RPA) which can efficiently amplify the target fragment to establish a CRISPR-Cas13a-based nucleic acid detection platform SHERLOCK (Specific High-Sensitivity Enzymatic Reporter UnLOCKing), with attomolar sensitivity and single-base specificity, allowing for rapid, inexpensive, and sensitive detection of trace nucleic acid. Studies have demonstrated that Cas13a can be used for detecting Zika and Dengue virus in biological samples (blood or urine), distinguish the genetic sequences of African and American strains, and identifying specific types of bacteria. After identifying viral or bacterial nucleic acids, Cas13a can be directly used for typing of pathogens by designing specific crRNA. Duce to the ultra-high sensitivity of Cas13, it can avoid a lot of complicated upstream experiments and allow for the directly amplifying biological samples for detection, thereby shortening the sample pretreatment process. As can be seen, this detection technology has great application prospects in the fields of basic research, diagnosis and treatment.

### SUMMARY

The present disclosure aims to solve at least one of the above-mentioned technical problems in the prior art. Thus, the present disclosure provides a CRISPR-Cas13 system for detecting SARS-CoV-2.

The present disclosure also provides a kit for detecting SARS-COV-2.

The present disclosure also provides use of the above-mentioned CRISPR-Cas13 system or kit.

In a first aspect, the present disclosure provides a CRISPR-Cas13 system for detecting SARS-CoV-2, comprising 1) or 2):
1) Cas13a protein and crRNA;
2) a complex formed by Cas13a protein and crRNA;
wherein the crRNA comprises a first guide RNA and a second guide RNA;
the first guide RNA and the second guide RNA comprise at least one sequence selected from the group consisting of SEQ ID NOs: 1 to 33, respectively.

In some embodiments, the crRNA is a guide sequence designed for a SARS-COV-2 target gene.

In some embodiments, the first guide RNA and the second guide RNA comprise different sequences; and the first guide RNA sequence and the second guide RNA sequence are used to cleave the same segment of the SARS-COV-2 target sequence.

In some embodiments, the first guide RNA and the second guide RNA comprise at least one sequence selected from the group consisting of SEQ ID NO:4, SEQ ID NO:5, SEQ ID NO:6, SEQ ID NO:7, SEQ ID NO:8, SEQ ID NO:9, SEQ ID NO:10, SEQ ID NO: 11, SEQ ID NO: 14, SEQ ID NO:15, SEQ ID NO:16, SEQ ID NO:17, SEQ ID NO:23 and SEQ ID NO:24, respectively.

In some embodiments, the SARS-COV-2 target gene is a spike protein.

In some embodiments, the SARS-COV-2 target sequence targeted by the crRNA comprises at least one sequence selected from the group consisting of SEQ ID NOs: 34 to 65.

In some embodiments, the CRISPR-Cas13 system further comprises a probe sequence, wherein the probe sequence comprises at least one sequence selected from the group consisting of SEQ ID NOs: 66 to 97.

In some embodiments, the probe sequence comprises at least one sequence selected from the group consisting of SEQ ID NO:69, SEQ ID NO:71, SEQ ID NO:73, SEQ ID NO:74, SEQ ID NO:75, SEQ ID NO:79, SEQ ID NO:80, SEQ ID NO:82 and SEQ ID NO:88.

In some embodiments, a crRNA-binding protospacer sequence comprises at least one sequence selected from the group consisting of SEQ ID NOs: 98 to 130.

In some embodiments, the Cas13a protein is LwCas13a protein.

In some embodiments, after the crRNA recognizes the target gene, the enzymatic activity of the Cas13a protein is activated to cleave the target RNA of the SARS-COV-2, with hybridizing the probe RNA with the target RNA fragment to release a detection signal.

In a second aspect, the present disclosure provides a kit for detecting SARS-COV-2, comprising the above-mentioned CRISPR-Cas13 system.

In some embodiments, the kit further comprises an RNA extraction kit, a LSPR biosensor, and a plasmonic waveguide microarray chip.

In a third aspect, the present disclosure provides use of the above-mentioned CRISPR-Cas13 system or kit, comprising any one of the following a1)-a3):
a1) for the preparation of products for detecting or aiding in the detection of SARS-COV-2;
a2) for the preparation of products for detecting or aiding in the detection of SARS-COV-2 nucleic acid;
a3) for the preparation of products for screening or aiding in the screening of drugs for preventing and treating SARS-COV-2.

In some embodiments, the method for detecting or aiding in the detection of SARS-COV-2 nucleic acid comprises the steps of:
(1) extracting nucleic acid from a sample to be tested;
(2) adding a CRISPR-Cas13 system, a probe, titanium nitride nanocubes and the nucleic acid from the sample to be tested to a plasma waveguide microarray chip and leaving it to stand for observation;
(3) using a LSPR biosensor for signal detection, if the LSPR biosensor detects a signal, the sample to be tested contains the SARS-COV-2 or is a candidate that contains the SARS-COV-2; if no signal is detected, the sample to be tested does not contain the SARS-COV-2 or is a candidate that does not contain the SARS-COV-2.

According to some embodiments, the present disclosure has at least the following beneficial effects.

By utilizing the guide RNA of the CRISPR/Cas13 system of the gene editing system, the present disclosure can accurately identify the specific RNA sequence comprising the spike protein gene in SARS-COV-2 and initiate Cas13 enzymatic activity, recognize and cleave viral target RNA, as well as detect SARS-COV-2 nucleic acid signals through thermoplasmonic amplification, titanium nitride nanocubes and thermoplasmonic chip microarray implementing multiple genes sensing. The scheme of the present disclosure is simple, sensitive, specific and accurate, and can be accurately used to detect the SARS-COV-2. Furthermore, the detection sensitivity of the scheme of the present disclosure is 8%-25% higher than that of the fluorescent quantitative polymerase chain reaction, and has a great application prospect.

### BRIEF DESCRIPTION OF THE DRAWINGS

The present disclosure is further described below in combination with drawings and embodiments.

FIG. 1 shows a schematic diagram of the detection principle of the SARS-COV-2 spike protein nucleic acid molecule in Example 2 of the present disclosure;

FIG. 2 shows the cleavage process repeated by Cas13a-crRNA_02 and Cas13a-crRNA_03 for the spike RNA fragment_02 in Example 2 of the present disclosure;

FIG. 3 shows that after cleavage of the spike RNA and production of fragments, each fragment is hybridized by the corresponding synthetic probes in Example 2 of the present disclosure;

FIG. 4 shows a structural diagram of the plasmonic waveguide microarray chip for detecting the spike protein RNA sequence in Example 2 of the present disclosure;

FIG. 5 shows the calculation results of the minimum free energy of the spike protein RNA target sequence (#21606 to #22441 as in Table 1) in Example 2 of the present disclosure;

FIG. 6 shows the calculation results of the minimum free energy of the spike protein RNA target sequence (#22546 to #23411 as in Table 1) in Example 2 of the present disclosure;

FIG. 7 shows the calculation results of the minimum free energy of the spike protein RNA target sequence (#23531 to #24371 as in Table 1) in Example 2 of the present disclosure;

FIG. 8 shows the calculation results of the minimum free energy of the spike protein RNA target sequence (#24491 to #25311 as in Table 1) in Example 2 of the present disclosure;

FIG. 9 shows the calculation results of the minimum free energy of complementary and reverse-complementary RNA to the spike protein target sequence (RNA probe_01 to RNA probe_08 as in Table 3) in Example 2 of the present disclosure;

FIG. 10 shows the calculation results of the minimum free energy of complementary and reverse-complementary RNA to the spike protein target sequence (RNA probe_09 to RNA probe_16 as in Table 3) in Example 2 of the present disclosure;

FIG. 11 shows the calculation results of the minimum free energy of complementary and reverse-complementary RNA to the spike protein target sequence (RNA probe_17 to RNA probe_24 as in Table 3) in Example 2 of the present disclosure;

FIG. 12 shows the calculation results of the minimum free energy of complementary and reverse-complementary RNA to the spike protein target sequence (RNA probe_25 to RNA probe_32 as in Table 3) in Example 2 of the present disclosure;

FIG. 13 shows the calculation results of the minimum free energy of crRNA (crRNA_01 to crRNA_08 as in Table 4) in Example 2 of the present disclosure;

FIG. 14 shows the calculation results of the minimum free energy of crRNA (crRNA_09 to crRNA_16 as in Table 4) in Example 2 of the present disclosure;

FIG. 15 shows the calculation results of the minimum free energy of crRNA (crRNA_17 to crRNA_24 as in Table 4) in Example 2 of the present disclosure;

FIG. 16 shows the calculation results of the minimum free energy of crRNA (crRNA_25 to crRNA_33 as in Table 4) in Example 2 of the present disclosure;

FIG. 17 shows the image of the 12×12 plasmonic microarray for sequential nucleic acid test of the SARS-COV-2 spike fragments by hybridization in Example 2 of the present disclosure;

FIG. 18 shows the secondary RNA structure of the complete spike gene with total of 3821 ribonucleic acid bases computed by the minimum free energy approach in Example 2 of the present disclosure;

FIG. 19 shows a diagram of the nucleic acid detection results of the G-04 flow cell in Example 3 of the present disclosure;

FIG. 20 shows a diagram of the nucleic acid detection results of the I-04 flow cell in Example 3 of the present disclosure;

FIG. 21 shows a diagram of the nucleic acid detection results of the E-05 flow cell in Example 3 of the present disclosure;

FIG. 22 shows a diagram of the nucleic acid detection results of the F-05 flow cell in Example 3 of the present disclosure;

FIG. 23 shows a diagram of the nucleic acid detection results of the G-05 flow cell in Example 3 of the present disclosure;

FIG. 24 shows a diagram of the nucleic acid detection results of the E-06 flow cell in Example 3 of the present disclosure;

FIG. 25 shows a diagram of the nucleic acid detection results of the F-06 flow cell in Example 3 of the present disclosure;

FIG. 26 shows a diagram of the nucleic acid detection results of the H-06 flow cell in Example 3 of the present disclosure;

FIG. 27 shows a diagram of the nucleic acid detection results of the H-07 flow cell in Example 3 of the present disclosure;

FIG. 28 shows the results of nucleic acid detection using a G-04 flow cell for nuclease-free water, 1 pM positive nucleic acid samples, and 1 nM positive nucleic acid samples in Example 3 of the present disclosure.

### DETAILED DESCRIPTION

The conception and technical effects of the present disclosure will be clearly and completely described below in conjunction with the embodiments, so as to fully understand the purpose, features and effects of the present disclosure. Apparently, the described embodiments are only some of the embodiments of the present disclosure, not all of them. Based on the embodiments of the present disclosure, other embodiments obtained by those skilled in the art without creative efforts are all within the protection scope of the present disclosure.

Reagents: Recombinant CRISPR-Cas13a protein (purchased from Beijing KEXIN Biomedical Technology Co., Ltd., China), titanium nitride nanocubes (purchased from US Research Nanomaterials, Inc.), plasmonic waveguide microarray chip (provided by RAFAEL BIOTECHNOLOGY COMPANY LIMITED), and SARS-COV-2 spike protein (purchased from Shanghai Beyotime Biotechnology Co., Ltd.).

### Example 1 a CRISPR-Cas13 System for Detecting the SARS-COV-2 Spike Protein

This example provided a CRISPR-Cas13a system for detecting the SARS-COV-2 spike protein, comprising crRNA, Cas13a protein and a probe.

### 1. Searching for CRISPR-Cas13a Nucleic Acid Detection Targets Based on the Gene Sequence Analysis of the SARS-COV-2 Spike Protein

Methods: The spike protein gene sequence was downloaded from the NCBI database, and the gene sequence alignment was conducted using the mafft software, with the automatic parameters set and conserved gene sequence. Moreover, the gene sequences of six other human-infecting coronaviruses, including HCoV-OC43, HCoV-229E, HCoV-NL63, HCoV-HKU1, SARS-COV and MERS-COV, were downloaded from the NCBI database, and the sequence alignment was conducted between the gene sequences of these coronaviruses and the conserved gene sequence of the spike protein to obtain the conserved gene sequence of the spike protein.

The nucleic acid detection target gene sequence of the CRISPR-Cas13a system has a non-G nucleotide at the 3′ end. The nucleic acid detection target of the CRISPR-Cas13a system was searched for in the conserved region of the spike protein gene sequence obtained by sequence alignment.

After the sequence alignment, the CRISPR-Cas13a nucleic acid detection target sequences (cleaved spike protein RNA target sequences) of 32 spike protein genes were obtained, and, as shown in Table 1, which did not overlap with the gene sequences of six other human-infecting coronaviruses.

Since the single-stranded gene RNA sequence of coronavirus can be folded freely to form a secondary structure and release energy (minimum free energy), the amount of released energy determines the stability of the secondary structure. The more negative the MFE, the more stable the secondary structure but the more difficult to hybridize with its complementary RNA sequence. The minimum free energy (MFE) of each target sequence in kcal/mol is as shown in Table 1.

TABLE 1

| Sequence | | MFE |
|---|---|---|
| number | Cleaved spike RNA targets (5′-3′) | (kcal/mol) |
| 21606- | GUGUUAAUCUUACAACCAGAACUCAAUUACCCCCUGCA | −0.74 |
| 21666 | UACACUAAUUCUUUCACACGUG (SEQ ID NO: 34) | |
| 21716- | CAGGACUUGUUCUUACCUUUCUUUUCCAAUGUUACUU | −6.20 |
| 21776 | GGUUCCAUGCUAUACAUGUCUCU (SEQ ID NO: 35) | |
| 21831- | UUUAUUUUGCUUCCACUGAGAAGUCUAACAUAAUAAG | −5.70 |
| 21891 | AGGCUGGAUUUUUGGUACUACUU (SEQ ID NO: 36) | |
| 21951- | UCUGUGAAUUUCAAUUUUGUAAUGAUCCAUUUUUGGG | −8.00 |
| 22011 | UGUUUAUUACCACAAAAACAACA (SEQ ID NO: 37) | |
| 22081- | GCCUUUUCUUAUGGACCUUGAAGGAAAACAGGGUAAU | −8.60 |
| 22141 | UUCAAAAAUCUUAGGGAAUUUGU (SEQ ID NO: 38) | |
| 22201- | GCGUGAUCUCCCUCAGGGUUUUUCGGCUUUAGAACCA | −15.80 |
| 22261 | UUGGUAGAUUUGCCAAUAGGUAU (SEQ ID NO: 39) | |
| 22316- | GGUGAUUCUUCUUCAGGUUGGACAGCUGGUGCUGCAG | −15.50 |
| 22376 | CUUAUUAUGUGGGUUAUCUUCAA (SEQ ID NO: 40) | |
| 22441- | UGACCCUCUCUCAGAAACAAAGUGUACGUUGAAAUCC | −4.90 |
| 22501 | UUCACUGUAGAAAAAGGAAUCUA (SEQ ID NO: 41) | |
| 22546- | AUUUCCUAAUAUUACAAACUUGUGCCCUUUUGGUGAA | −9.40 |
| 22606 | GUUUUUAACGCCACCAGAUUUGC (SEQ ID NO: 42) | |
| 22676- | GCAUCAUUUUCCACUUUUAAGUGUUAUGGAGUGUCUC | −7.60 |
| 22736 | CUACUAAAUUAAAUGAUCUCUGC (SEQ ID NO: 43) | |
| 22806- | CUGGAAAGAUUGCUGAUUAUAAUUAUAAAUUACCAGA | −5.80 |
| 22866 | UGAUUUUACAGGCUGCGUUAUAG (SEQ ID NO: 44) | |
| 22916- | CUGUAUAGAUUGUUUAGGAAGUCUAAUCUCAAACCUU | −10.20 |
| 22976 | UUGAGAGAGAUAUUUCAACUGAA (SEQ ID NO: 45) | |
| 23046- | AUGGUUUCCAACCCACUAAUGGUGUUGGUUACCAACC | −18.60 |
| 23106 | AUACAGAGUAGUAGUACUUUCUU (SEQ ID NO: 46) | |
| 23171- | AAAUGUGUCAAUUUCAACUUCAAUGGUUUAACAGGCA | −8.00 |
| 23231 | CAGGUGUUCUUACUGAGUCUAAC (SEQ ID NO: 47) | |
| 23291- | CGUGAUCCACAGACACUUGAGAUUCUUGACAUUACACC | −15.10 |
| 23351 | AUGUUCUUUUGGUGGUGUCAGU (SEQ ID NO: 48) | |
| 23411- | UGCACAGAAGUCCCUGUUGCUAUUCAUGCAGAUCAAC | −9.40 |
| 23471 | UUACUCCUACUUGGCGUGUUUAU (SEQ ID NO: 49) | |
| 23531- | AACAACUCAUAUGAGUGUGACAUACCCAUUGGUGCAG | −14.10 |
| 23591 | GUAUAUGCGCUAGUUAUCAGACU (SEQ ID NO: 50) | |
| 23651- | AUGUCACUUGGUGCAGAAAAUUCAGUUGCUUACUCUA | −4.10 |
| 23711 | AUAACUCUAUUGCCAUACCCACA (SEQ ID NO: 51) | |
| 23761- | GACAUCAGUAGAUUGUACAAUGUACAUUUGUGGUGAU | −9.40 |
| 23821 | UCAACUGAAUGCAGCAAUCUUUU (SEQ ID NO: 52) | |
| 23876- | GUUGAACAAGACAAAAACACCCAAGAAGUUUUUGCAC | −6.10 |
| 23936 | AAGUCAAACAAAUUUACAAAACA (SEQ ID NO: 53) | |
| 23996- | CCAAGCAAGAGGUCAUUUAUUGAAGAUCUACUUUUCA | −17.60 |
| 23456 | ACAAAGUGACACUUGCAGAUGCU (SEQ ID NO: 54) | |
| 24116- | GCACAAAAGUUUAACGGCCUUACUGUUUUGCCACCUU | −5.40 |
| 24176 | UGCUCACAGAUGAAAUGAUUGCU (SEQ ID NO: 55) | |
| 24241- | AUUACAAAUACCAUUUGCUAUGCAAAUGGCUUAUAGG | −11.50 |
| 24301 | UUUAAUGGUAUUGGAGUUACACA (SEQ ID NO: 56) | |
| 24371- | UCACUUUCUUCCACAGCAAGUGCACUUGGAAAACUUCA | −8.80 |
| 24431 | AGAUGUGGUCAACCAAAAUGCA (SEQ ID NO: 57) | |
| 24491- | UUAAAUGAUAUCCUUUCACGUCUUGACAAAGUUGAGG | −9.80 |
| 24551 | CUGAAGUGCAAAUUGAUAGGUUG (SEQ ID NO: 58) | |
| 24601- | UAGAGCUGCAGAAAUCAGAGCUUCUGCUAAUCUUGCU | −11.30 |
| 24661 | GCUACUAAAAUGUCAGAGUGUGU (SEQ ID NO: 59) | |
| 24721- | UCAGUCAGCACCUCAUGGUGUAGUCUUCUUGCAUGUG | −14.70 |
| 24781 | ACUUAUGUCCCUGCACAAGAAAA (SEQ ID NO: 60) | |
| 24841- | UGUCUUUGUUUCAAAUGGCACACACUGGUUUGUAACA | −9.60 |
| 24901 | CAAAGGAAUUUUUAUGAACCACA (SEQ ID NO: 61) | |
| 24961- | AUUGUCAACAACACAGUUUAUGAUCCUUUGCAACCUG | −5.40 |
| 25021 | AAUUAGACUCAUUCAAGGAGGAG (SEQ ID NO: 62) | |
| 25081- | UGCUUCAGUUGUAAACAUUCAAAAAGAAAUUGACCGC | −7.00 |
| 25141 | CUCAAUGAGGUUGCCAAGAAUUU (SEQ ID NO: 63) | |
| 25201- | GAGCAGUAUAUAAAAUGGCCAUGGUACAUUUGGCUAG | −16.40 |
| 25261 | GUUUUAUAGCUGGCUUGAUUGCC (SEQ ID NO: 64) | |
| 25311- | AGUUGUCUCAAGGGCUGUUGUUCUUGUGGAUCCUGCU | −13.80 |
| 25371 | GCAAAUUUGAUGAAGACGACUCU (SEQ ID NO: 65) | |

### 2. Obtain Protospacer Sequences

According to the CRISPR-Cas13a nucleic acid detection target sequences of 32 spike protein genes, 33protospacers of nucleic acid detection targets were determined, as shown in Table 2. The MPE of each protospacer in kcal/mol is shown in Table 2.

TABLE 2

| | Sequence | Protospacer on the spike RNA | MFE |
|---|---|---|---|
| Name | number | sequence (5′-3′) | (kcal/mol) |
| Protospacer | 21568- | UGUUUUUCUUGUUUUAUUGCCACUAGU | 0.00 |
| 01 | 21596 | CU (SEQ ID NO: 98) | |
| Protospacer | 21675- | ACCCUGACAAAGUUUUCAGAUCCUCAG | −2.90 |
| 02 | 21703 | UU (SEQ ID NO: 99) | |
| Protospacer | 21785- | GGUACUAAGAGGUUUGAUAACCCUGUC | −5.30 |
| 03 | 21813 | CU (SEQ ID NO: 100) | |
| Protospacer | 21903- | CCCAGUCCCUACUUAUUGUUAAUAACG | −0.00 |
| 04 | 21931 | CU (SEQ ID NO: 101) | |
| Protospacer | 22026- | GUGAGUUCAGAGUUUAUUCUAGUGCGA | −0.10 |
| 05 | 22054 | AU (SEQ ID NO: 102) | |
| Protospacer | 22172- | UAUUCUAAGCACACGCCUAUUAAUUUA | −0.50 |
| 06 | 22200 | GU (SEQ ID NO: 103) | |
| Protospacer | 22264- | CAUCACUAGGUUUCAAACUUUACUUGC | −0.50 |
| 07 | 22292 | UU (SEQ ID NO: 104) | |
| Protospacer | 22499- | GGAACCAUUACAGAUGCUGUAGACUGU | −2.80 |
| 08 | 22527 | GC (SEQ ID NO: 105) | |
| Protospacer | 22505- | ACUUCUAACUUUAGAGUCCAACCAACA | −1.60 |
| 09 | 22533 | GA (SEQ ID NO: 106) | |
| Protospacer | 22638- | GCAACUGUGUUGCUGAUUAUUCUGUCC | −4.50 |
| 10 | 22666 | UA (SEQ ID NO: 107) | |
| Protospacer | 22770- | GAGGUGAUGAAGUCAGACAAAUCGCUC | −4.50 |
| 11 | 22798 | CA (SEQ ID NO: 108) | |
| Protospacer | 22875- | CUAACAAUCUUGAUUCUAAGGUUGGUG | −7.40 |
| 12 | 22903 | GU (SEQ ID NO: 109) | |
| Protospacer | 22998- | CUUGUAAUGGUGUUGAAGGUUUUAAU | 0.00 |
| 13 | 23026 | UGU (SEQ ID NO: 110) | |
| Protospacer | 23125- | AGCAACUGUUUGUGGACCUAAAAAGUC | −2.40 |
| 14 | 23153 | UA (SEQ ID NO: 111) | |
| Protospacer | 23248- | CCAACAAUUUGGCAGAGACAUUGCUGA | −3.50 |
| 15 | 23276 | CA (SEQ ID NO: 112) | |
| Protospacer | 23364- | GAACAAAUACUUCUAACCAGGUUGCUG | −1.90 |
| 16 | 23392 | UU (SEQ ID NO: 113) | |
| Protospacer | 23482- | UAAUGUUUUUCAAACACGUGCAGGCUG | −1.30 |
| 17 | 23510 | UU (SEQ ID NO: 114) | |
| Protospacer | 23609- | CGGGCACGUAGUGUAGCUAGUCAAUCC | −3.10 |
| 18 | 23637 | AU (SEQ ID NO: 115) | |
| Protospacer | 23723- | AGUGUUACCACAGAAAUUCUACCAGUG | −1.20 |
| 19 | 23751 | UC (SEQ ID NO: 116) | |
| Protospacer | 23831- | GGCAGUUUUUGUACACAAUUAAACCGU | −1.40 |
| 20 | 23859 | GC (SEQ ID NO: 117) | |
| Protospacer | 23954- | GGUGGUUUUAAUUUUUCACAAAUAUUA | −2.20 |
| 21 | 23982 | CC (SEQ ID NO: 118) | |
| Protospacer | 24059- | AAUAUGGUGAUUGCCUUGGUGAUAUUG | −1.70 |
| 22 | 24087 | CU (SEQ ID NO: 19) | |
| Protospacer | 24194- | UUAGCGGGUACAAUCACUUCUGGUUGG | −4.20 |
| 23 | 24222 | AC (SEQ ID NO: 120) | |
| Protospacer | 24325- | AUUGAUUGCCAACCAAUUUAAUAGUGC | −0.40 |
| 24 | 24353 | UA (SEQ ID NO: 121) | |
| Protospacer | 24447- | UUGUUAAACAACUUAGCUCCAAUUUUG | −2.30 |
| 25 | 24475 | GU (SEQ ID NO: 122) | |
| Protospacer | 24550- | AGACUUCAAAGUUUGCAGACAUAUGUG | −1.70 |
| 26 | 24578 | AC (SEQ ID NO: 123) | |
| Protospacer | 24668- | CAAUCAAAAAGAGUUGAUUUUUGUGGA | −2.40 |
| 27 | 24696 | AA (SEQ ID NO: 124) | |
| Protospacer | 24801- | CCAUUUGUCAUGAUGGAAAAGCACACU | −4.40 |
| 28 | 24829 | UU (SEQ ID NO: 125) | |
| Protospacer | 24915- | ACAACACAUUUGUGUCUGGUAACUGUG | −3.20 |
| 29 | 24943 | AU (SEQ ID NO: 126) | |
| Protospacer | 25029- | UUAAGAAUCAUACAUCACCAGAUGUUG | −6.20 |
| 30 | 25057 | AU (SEQ ID NO: 127) | |
| Protospacer | 25146- | AAUCUCUCAUCGAUCUCCAAGAACUUG | −3.20 |
| 31 | 25174 | GA (SEQ ID NO: 128) | |
| Protospacer | 25199- | GGUGACAAUUAUGCUUUGCUGUAUGAC | −4.10 |
| 32 | 25227 | CA (SEQ ID NO: 129) | |
| Protospacer | 25356- | UCAAAGGAGUCAAAUUACAUUACACAU | 0.00 |
| 33 | 25384 | AA (SEQ ID NO: 130) | |

### 3. Design and Synthesis of Probe Sequence

According to the CRISPR-Cas13a nucleic acid detection target sequences of 32 spike protein genes, 32 complementary hybridization probe sequences in length 60 nucleotides, as shown in Table 3, were designed and fabricated by Sangon Biotech Co., Ltd (Shanghai, China). The MFE of each probe sequence in kcal/mol is shown in Table 3.

TABLE 3

| | Sequence | | MFE |
|---|---|---|---|
| Name | number | Probe sequence (5′-3′) | (kcal/mol) |
| RNA | 21606- | CACGUGUGAAAGAAUUAGUGUAUGCAGGGG | −4.80 |
| probe_01 | 21666 | GUAAUUGAGUUCUGGUUGUAAGAUUAACAC | |
| | | (SEQ ID NO: 66) | |
| RNA | 21716- | GUCCUGAACAAGAAUGGAAAGAAAAGGUUA | −7.40 |
| probe_02 | 21776 | CAAUGAACCAAGGUACGAUAUGUACAGAGA | |
| | | (SEQ ID NO: 67) | |
| RNA | 21831- | AAAUAAAACGAAGGUGACUCUUCAGAUUGU | −3.20 |
| probe_03 | 21891 | AUUAUUCUCCGACCUAAAAACCAUGAUGAA | |
| | | (SEQ ID NO: 68) | |
| RNA | 21951- | AGACACUUAAAGUUAAAACAUUACUAGGUA | −5.90 |
| probe_04 | 22011 | AAAACCCACAAAUAAUGGUGUUUUUGUUGU | |
| | | (SEQ ID NO: 69) | |
| RNA | 22081- | ACAAAUUCCCUAAGAUUUUUGAAAUUACCC | −4.10 |
| probe_05 | 22141 | UGUUUUCCUUCAAGGUCCAUAAGAAAAGGC | |
| | | (SEQ ID NO: 70) | |
| RNA | 22201- | AUACCUAUUGGCAAAUCUACCAAUGGUUCU | −9.40 |
| probe_06 | 22261 | AAAGCCGAAAAACCCUGAGGGAGAUCACGC | |
| | | (SEQ ID NO: 71) | |
| RNA | 22316- | CCACUAAGAAGAAGUCCAACCUGUCGACCA | −5.40 |
| probe_07 | 22376 | CGACGUCGAAUAAUACACCCAAUAGAAGUU | |
| | | (SEQ ID NO: 72) | |
| RNA | 22441- | ACUGGGAGAGAGUCUUUGUUUCACAUGCAA | −10.70 |
| probe_08 | 22501 | CUUUAGGAAGUGACAUCUUUUUCCUUAGAU | |
| | | (SEQ ID NO: 73) | |
| RNA | 22546- | UAAAGGAUUAUAAUGUUUGAACACGGGAA | −9.20 |
| probe_09 | 22606 | AACCACUUCAAAAAUUGCGGUGGUCUAAAC | |
| | | G (SEQ ID NO: 74) | |
| RNA | 22676- | CGUAGUAAAAGGUGAAAAUUCACAAUACCU | −9.20 |
| probe_10 | 22736 | CACAGAGGAUGAUUUAAUUUACUAGAGACG | |
| | | (SEQ ID NO: 75) | |
| RNA | 22806- | CUAUAACGCAGCCUGUAAAAUCAUCUGGUA | −3.60 |
| probe_11 | 22866 | AUUUAUAAUUAUAAUCAGCAAUCUUUCCAG | |
| | | (SEQ ID NO: 76) | |
| RNA | 22916- | GACAUAUCUAACAAAUCCUUCAGAUUAGAG | −5.80 |
| probe_12 | 22976 | UUUGGAAAACUCUCUCUAUAAAGUUGACUU | |
| | | (SEQ ID NO: 77) | |
| RNA | 23046- | UACCAAAGGUUGGGUGAUUACCACAACCAA | −14.40 |
| probe_13 | 23106 | UGGUUGGUAUGUCUCAUCAUCAUGAAAGAA | |
| | | (SEQ ID NO: 78) | |
| RNA | 23171- | GUUAGACUCAGUAAGAACACCUGUGCCUGU | −5.30 |
| probe_14 | 23231 | UAAACCAUUGAAGUUGAAAUUGACACAUUU | |
| | | (SEQ ID NO: 79) | |
| RNA | 23291- | GCACUAGGUGUCUGUGAACUCUAAGAACUG | −12.50 |
| probe_15 | 23351 | UAAUGUGGUACAAGAAAACCACCACAGUCA | |
| | | (SEQ ID NO: 80) | |
| RNA | 23411- | AUAAACACGCCAAGUAGGAGUAAGUUGAUC | −8.80 |
| probe_16 | 23471 | UGCAUGAAUAGCAACAGGGACUUCUGUGCA | |
| | | (SEQ ID NO: 81) | |
| RNA | 23531- | UUGUUGAGUAUACUCACACUGUAUGGGUAA | −9.30 |
| probe_17 | 23591 | CCACGUCCAUAUACGCGAUCAAUAGUCUGA | |
| | | (SEQ ID NO: 82) | |
| RNA | 23651- | UGUGGGUAUGGCAAUAGAGUUAUUAGAGU | −6.40 |
| probe_18 | 23711 | AAGCAACUGAAUUUUCUGCACCAAGUGACA | |
| | | U (SEQ ID NO: 83) | |
| RNA | 23761- | CUGUAGUCAUCUAACAUGUUACAUGUAAAC | −8.50 |
| probe_19 | 23821 | ACCACUAAGUUGACUUACGUCGUUAGAAAA | |
| | | (SEQ ID NO: 84) | |
| RNA | 23876- | CAACUUGUUCUGUUUUUGUGGGUUCUUCAA | −6.90 |
| probe_20 | 23936 | AAACGUGUUCAGUUUGUUUAAAUGUUUUG | |
| | | U (SEQ ID NO: 85) | |
| RNA | 23996- | GGUUCGUUCUCCAGUAAAUAACUUCUAGAU | −15.50 |
| probe_21 | 23456 | GAAAAGUUGUUUCACUGUGAACGUCUACGA | |
| | | (SEQ ID NO: 86) | |
| RNA | 24116- | CGUGUUUUCAAAUUGCCGGAAUGACAAAAC | −9.00 |
| probe_22 | 24176 | GGUGGAAACGAGUGUCUACUUUACUAACGA | |
| | | (SEQ ID NO: 87) | |
| RNA | 24241- | UAAUGUUUAUGGUAAACGAUACGUUUACCG | −10.40 |
| probe_23 | 24301 | AAUAUCCAAAUUACCAUAACCUCAAUGUGU | |
| | | (SEQ ID NO: 88) | |
| RNA | 24371- | AGUGAAAGAAGGUGUCGUUCACGUGAACCU | −9.90 |
| probe_24 | 24431 | UUUGAAGUUCUACACCAGUUGGUUUUACGU | |
| | | (SEQ ID NO89) | |
| RNA | 24491- | CAACCUAUCAAUUUGCACUUCAGCCUCAAC | −5.00 |
| probe_25 | 24551 | UUUGUCAAGACGUGAAAGGAUAUCAUUUAA | |
| | | (SEQ ID NO: 90) | |
| RNA | 24601- | AUCUCGACGUCUUUAGUCUCGAAGACGAUU | −10.60 |
| probe_26 | 24661 | AGAACGACGAUGAUUUUACAGUCUCACACA | |
| | | (SEQ ID NO: 91) | |
| RNA | 24721- | AGUCAGUCGUGGAGUACCACAUCAGAAGAA | −13.20 |
| probe_27 | 24781 | CGUACACUGAAUACAGGGACGUGUUCUUUU | |
| | | (SEQ ID NO: 92) | |
| RNA | 24841- | ACAGAAACAAAGUUUACCGUGUGUGACCAA | −6.20 |
| probe_28 | 24901 | ACAUUGUGUUUCCUUAAAAAUACUUGGUGU | |
| | | (SEQ ID NO: 93) | |
| RNA | 24961- | UAACAGUUGUUGUGUCAAAUACUAGGAAAC | −6.40 |
| probe_29 | 25021 | GUUGGACUUAAUCUGAGUAAGUUCCUCCUC | |
| | | (SEQ ID NO: 94) | |
| RNA | 25081- | ACGAAGUCAACAUUUGUAAGUUUUUCUUUA | −8.60 |
| probe_30 | 25141 | ACUGGCGGAGUUACUCCAACGGUUCUUAAA | |
| | | (SEQ ID NO: 95) | |
| RNA | 25201- | CUCGUCAUAUAUUUUACCGGUACCAUGUAA | −5.90 |
| probe_31 | 25261 | ACCGAUCCAAAAUAUCGACCGAACUAACGG | |
| | | (SEQ ID NO: 96) | |
| RNA | 25311- | AGAGUCGUCUUCAUCAAAUUUGCAGCAGGA | −4.00 |
| probe_32 | 25371 | UCCACAAGAACAACAGCCCUUGAGACAACU | |
| | | (SEQ ID NO: 97) | |

4. Design and Synthesis of crRNA for Each Detection Target

According to the gene sequence of each target, the protospacer sequence of the CRISPR-Cas13a nucleic acid detection targets and the CRISPR-Cas13a nucleic acid detection system, 33 pieces of synthetic crRNA with hairpin loop of 57 nucleotides in length were designed for specific cleavage of spike RNA. The crRNA sequence consisted of two parts: the conserved gene sequence at the 5′ end and the complementary sequence of the target gene sequence at the 3′ end. The crRNA sequence as shown in Table 4 was directly synthesized by Sangon Biotech Co., Ltd (Shanghai, China). The MFE of each crRNA sequence in kcal/mo is shown in Table 4.

TABLE 4

| | Sequence | crRNA sequence with hairpin | MFE |
|---|---|---|---|
| Name | number | loop (5′-3′) | (kcal/mol) |
| crRNA_01 | 21568- | ACAAAAAGAACAAAAUAACGGUGAUCAG | −6.70 |
| | 21596 | ACAAAAUCAGGGGAAGCUAUAACCCCACC | |
| | | (SEQ ID NO: 1) | |
| crRNA_02 | 21675- | UGGGACUGUUUCAAAAGUCUAGGAGUCA | −11.50 |
| | 21703 | ACAAAAUCAGGGGAAGCUAUAACCCCACC | |
| | | (SEQ ID NO: 2) | |
| crRNA_03 | 21785- | CCAUGAUUCUCCAAACUAUUGGGACAGGA | −10.50 |
| | 21813 | CAAAAUCAGGGGAAGCUAUAACCCCACC | |
| | | (SEQ ID NO: 3) | |
| crRNA_04 | 21903- | GGGUCAGGGAUGAAUAACAAUUAUUGCG | −8.50 |
| | 21931 | ACAAAAUCAGGGGAAGCUAUAACCCCACC | |
| | | (SEQ ID NO: 4) | |
| crRNA_05 | 22026- | CACUCAAGUCUCAAAUAAGAUCACGCUUA | −7.00 |
| | 22054 | CAAAAUCAGGGGAAGCUAUAACCCCACC | |
| | | (SEQ ID NO: 5) | |
| crRNA_06 | 22172- | AUAAGAUUCGUGUGCGGAUAAUUAAAUC | −8.50 |
| | 22200 | ACAAAAUCAGGGGAAGCUAUAACCCCACC | |
| | | (SEQ ID NO: 6) | |
| crRNA_07 | 22264- | GUAGUGAUCCAAAGUUUGAAAUGAACGA | −8.70 |
| | 22292 | ACAAAAUCAGGGGAAGCUAUAACCCCACC | |
| | | (SEQ ID NO: 7) | |
| crRNA_08 | 22499- | CCUUGGUAAUGUCUACGACAUCUGACACG | −9.00 |
| | 22527 | CAAAAUCAGGGGAAGCUAUAACCCCACC | |
| | | (SEQ ID NO: 8) | |
| crRNA_09 | 22505- | UGAAGAUUGAAAUCUCAGGUUGGUUGUC | −9.20 |
| | 22533 | UCAAAAUCAGGGGAAGCUAUAACCCCACC | |
| | | (SEQ ID NO: 9) | |
| crRNA 10 | 22638- | CGUUGACACAACGACUAAUAAGACAGGA | −9.40 |
| | 22666 | UCAAAAUCAGGGGAAGCUAUAACCCCACC | |
| | | (SEQ ID NO: 10) | |
| crRNA_11 | 22770- | CUCCACUACUUCAGUCUGUUUAGCGAGGU | −8.70 |
| | 22798 | CAAAAUCAGGGGAAGCUAUAACCCCACC | |
| | | (SEQ ID NO: 11) | |
| crRNA 12 | 22875- | GAUUGUUAGAACUAAGAUUCCAACCACCA | −6.90 |
| | 22903 | CAAAAUCAGGGGAAGCUAUAACCCCACC | |
| | | (SEQ ID NO: 12) | |
| crRNA_13 | 22998- | GAACAUUACCACAACUUCCAAAAUUAACA | −6.10 |
| | 23026 | CAAAAUCAGGGGAAGCUAUAACCCCACC | |
| | | (SEQ ID NO: 13) | |
| crRNA 14 | 23125- | UCGUUGACAAACACCUGGAUUUUUCAGA | −9.20 |
| | 23153 | UCAAAAUCAGGGGAAGCUAUAACCCCACC | |
| | | (SEQ ID NO: 14) | |
| crRNA_15 | 23248- | GGUUGUUAAACCGUCUCUGUAACGACUG | −12.00 |
| | 23276 | UCAAAAUCAGGGGAAGCUAUAACCCCACC | |
| | | (SEQ ID NO: 15) | |
| crRNA_16 | 23364- | CUUGUUUAUGAAGAUUGGUCCAACGACA | −7.70 |
| | 23392 | ACAAAAUCAGGGGAAGCUAUAACCCCACC | |
| | | (SEQ ID NO: 16) | |
| crRNA_17 | 23482- | AUUACAAAAAGUUUGUGCACGUCCGACA | −7.50 |
| | 23510 | ACAAAAUCAGGGGAAGCUAUAACCCCACC | |
| | | (SEQ ID NO: 17) | |
| crRNA_18 | 23609- | GCCCGUGCAUCACAUCGAUCAGUUAGGUA | −9.60 |
| | 23637 | CAAAAUCAGGGGAAGCUAUAACCCCACC | |
| | | (SEQ ID NO: 18) | |
| crRNA_19 | 23723- | UCACAAUGGUGUCUUUAAGAUGGUCACA | −9.40 |
| | 23751 | GCAAAAUCAGGGGAAGCUAUAACCCCACC | |
| | | (SEQ ID NO: 19) | |
| crRNA_20 | 23831- | CCGUCAAAAACAUGUGUUAAUUUGGCAC | −10.70 |
| | 23859 | GCAAAAUCAGGGGAAGCUAUAACCCCACC | |
| | | (SEQ ID NO: 20) | |
| crRNA_21 | 23954- | CCACCAAAAUUAAAAAGUGUUUAUAAUG | −7.00 |
| | 23982 | GCAAAAUCAGGGGAAGCUAUAACCCCACC | |
| | | (SEQ ID NO: 21) | |
| crRNA_22 | 24059- | UUAUACCACUAACGGAACCACUAUAACGA | −6.60 |
| | 24087 | CAAAAUCAGGGGAAGCUAUAACCCCACC | |
| | | (SEQ ID NO: 22) | |
| crRNA_23 | 24194- | AAUCGCCCAUGUUAGUGAAGACCAACCUG | −8.20 |
| | 24222 | CAAAAUCAGGGGAAGCUAUAACCCCACC | |
| | | (SEQ ID NO: 23) | |
| crRNA 24 | 24325- | UAACUAACGGUUGGUUAAAUUAUCACGA | −9.00 |
| | 24353 | UCAAAAUCAGGGGAAGCUAUAACCCCACC | |
| | | (SEQ ID NO: 24) | |
| crRNA_25 | 24447- | AACAAUUUGUUGAAUCGAGGUUAAAACC | −7.70 |
| | 24475 | ACAAAAUCAGGGGAAGCUAUAACCCCACC | |
| | | (SEQ ID NO: 25) | |
| crRNA_26 | 24550- | UCUGAAGUUUCAAACGUCUGUAUACACU | −6.80 |
| | 24578 | GCAAAAUCAGGGGAAGCUAUAACCCCACC | |
| | | (SEQ ID NO: 26) | |
| crRNA 27 | 24668- | GUUAGUUUUUCUCAACUAAAAACACCUU | −8.50 |
| | 24696 | UCAAAAUCAGGGGAAGCUAUAACCCCACC | |
| | | (SEQ ID NO: 27) | |
| crRNA_28 | 24801- | GGUAAACAGUACUACCUUUUCGUGUGAA | −9.40 |
| | 24829 | ACAAAAUCAGGGGAAGCUAUAACCCCACC | |
| | | (SEQ ID NO: 28) | |
| crRNA 29 | 24915- | UGUUGUGUAAACACAGACCAUUGACACU | −9.60 |
| | 24943 | ACAAAAUCAGGGGAAGCUAUAACCCCACC | |
| | | (SEQ ID NO: 29) | |
| crRNA_30 | 25029- | AAUUCUUAGUAUGUAGUGGUCUACAACU | −12.70 |
| | 25057 | ACAAAAUCAGGGGAAGCUAUAACCCCACC | |
| | | (SEQ ID NO: 30) | |
| crRNA_31 | 25146- | UUAGAGAGUAGCUAGAGGUUCUUGAACC | −13.10 |
| | 25174 | UCAAAAUCAGGGGAAGCUAUAACCCCACC | |
| | | (SEQ ID NO: 31) | |
| crRNA 32 | 25199- | CCACUGUUAAUACGAAACGACAUACUGGU | −8.20 |
| | 25227 | CAAAAUCAGGGGAAGCUAUAACCCCACC | |
| | | (SEQ ID NO: 32) | |
| crRNA_33 | 25356- | AGUUUCCUCAGUUUAAUGUAAUGUGUAU | −10.30 |
| | 25384 | UCAAAAUCAGGGGAAGCUAUAACCCCACC | |
| | | (SEQ ID NO: 33) | |

### Example 2

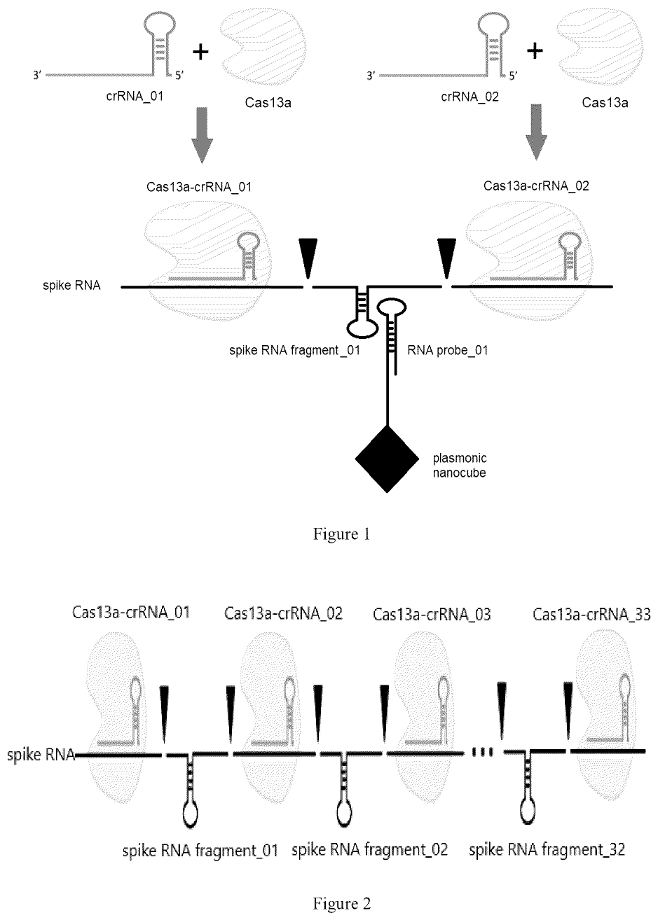

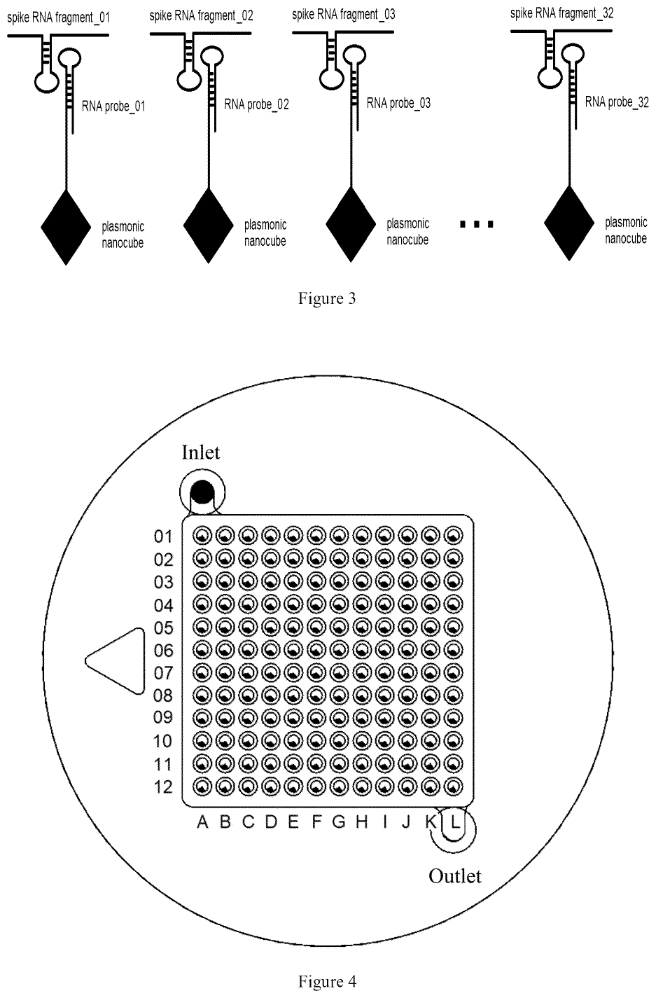

In this example, the nucleic acid molecule of the SARS-COV-2 spike protein was detected, and FIGS. 1 to 3 illustrate the scheme of the present disclosure. In FIG. 1, the Cas13a enzymes were incubated with different crRNA designated to respective spike segments. After incubation, the Cas13a-crRNA complexes in pair of two consecutive order, i.e., Cas13a-crRNA_01 and Cas13a-crRNA_02 were placed together in a plasmonic biosensing flow cell. On presences of the spike RNA sequence, the Cas13a armed with crRNA was activated. The Cas13a-crRNA_01 enzyme cleaved the spike RNA to produce the first cut of the spike RNA fragment_01. The Cas13a-crRNA_02 also cleaved the spike RNA to produce the second cut. The two cleavage sites were indicated by the two inverted triangles in FIG. 1. Therefore, spike RNA fragment_01 would be released from the spike sequence. Since Cas13a was highly specific to the crRNA target, cleavage actions were guaranteed to occur at the two targeted sites. The spike RNA fragment was a short single-strand RNA and it was detected by the synthetic complementary (or reverse-complementary) probes by hybridization. The probe was functionalized on the plasmonic nanocubes and the fragment-probe hybridization generated localized refractive index change on the plasmonic nanocubes. The plasmonic signal was registered by optical phase change in the plasmonic biosensor system. The cleavage process was repeated by Cas13a-crRNA_02 and Cas13a-crRNA_03 for the spike RNA fragment_02 and so on as illustrated by FIG. 2. In FIG. 2, there were total of 33 crRNA (SEQ ID NO: 1-33) which were complementary to the protospacer and the protospacer flanking sequence (PFS) present on the spike RNA sequence. The selection of protospacer and design of the crRNA was obtained by computational method. After cleavage of the spike RNA and production of fragments, each fragment was hybridized by the corresponding synthetic probes functionalized on the plasmonic nanocubes as depicted in FIG. 3. As these functionalized plasmonic nanocubes were anchored on different flow cells of our microarray biochip, the viral spike RNA was detected with high sensitivity and specificity.

The operation protocol was shown in the following steps:

(1) Total viral RNA of SARS-COV-2 was extracted with commercial RNA extraction kit, i.e., QIAwave RNA Mini Kit, Qiagen.

(2) The commercial purified LwaCas13a protein was diluted to 1 μM in a buffer which consisted of 20 mM HEPES (pH 7.5), 150 mM KCl, 10 mM MgCl₂ and 0.5 mM DTT. LwaCas13a (1 μM) was mixed with an equal volume of the crRNA (625 nM) in nuclease-free water, and then incubated at 37° ° C. for 10 min to obtain 33 sets of LwaCas13a-crRNA complexes.

(3) 1 uM synthetic RNA probes in the same buffer were prepared. There were 32 sets of RNA probes, and each set referred to corresponding probe listed in Table 3. 50 nanoliters of each RNA probes were dispensed into separate flow cell of the plasmonic waveguide microarray.

(4) Two consecutive LwaCas13a-crRNA complexes of 25 nanoliters each were dispensed into the flow cell containing the corresponding probe RNA on the plasmonic waveguide microarray. The LwaCas13a-crRNA-probe combination was referred to FIGS. 1 to 3. So, the first plasmonic flow cell contained titanium nitride nanocubes functionalized with RNA probe_01, LwaCas13a-crRNA_01 and LwaCas13a-crRNA_02. The second flow cell contained RNA probe_02, LwaCas13a-crRNA_02 and LwaCas13a-crRNA_03. There were 32 sets, and the arrangement of functionalization chemicals on the plasmonic waveguide microarrays was listed in Table 5. Functionalization referred to incubation at 37° C. for 10 minutes, pre-preformed prior to detection.

(5) 50 microliters of extracted viral RNA solution was injected into the plasmonic waveguide microarray which contained 144 flow cells and 32 of these were functionalized with respective RNA probe, LwaCas13a-crRNA complexes, and titanium nitride nanocubes as signal transducer.

(6) 50 microliters of RNA free buffer was used to flush the plasmonic waveguide microarray to ensure specific binding of the RNA probes to corresponding spike RNA fragments. Spike RNA concentration was measured by the optical phase change registered on the LSPR biosensor. Image of the plasmonic waveguide microarray as shown in FIG. 4 contained 144 flow cells in a 12-by-12 configuration for functionalization with RNA-probes and LwaCas13a-crRNA complexes. The inlet and outlet for viral RNA sample injection were also included. The plasmonic microarray was fabricated by direct 3D printing with poly(methyl methacrylate) (PMMA). The MFE of cleaved spike RNA target, probe sequence, crRNA sequence and the protospacer on the spike RNA sequence were calculated, and the change in MFE upon crRNA-protospacer hybridization, target-probe hybridization and probe was also calculated.

To further explore nonspecific hybridization of the complementary probe towards the crRNA, it is necessary to calculate the change in MFE assuming nonspecific hybridization occurs between the probe and crRNA. The detection steps were consistent with the above steps (1) to (6).

The interaction free energy approach was adopted for the analysis, and this was defined as the change minimum free energy (MFE) before and after hybridization. This was a benchmark to justify the preferential interaction between the sequences in the solution mixture. All calculations were done via the ViennaRNA Package 2.0. The change in energy was expressed by the Equation

$$Δ E = {E}_{h} - {E}_{rna 1} - {E}_{rna 2} ,$$

where ΔE is the change in MFE, Eₕ is the MFE of the two RNA after hybridization, Eᵣₙₐ₁ is the MFE of the first RNA, and Eᵣₙₐ₂ is the MFE of the second RNA.

Minimum free energy is defined as the energy required to change the RNA structure from its most thermodynamically stable secondary state to single stranded state.

TABLE 5

| Cell ID | |
|---|---|
| on microarray | Chemicals |
| D-04 | RNA probe_01, LwaCas13a-crRNA_01 and |
| | LwaCas13a-crRNA_02 |
| E-04 | RNA probe_02, LwaCas13a-crRNA_02 and |
| | LwaCas13a-crRNA_03 |
| F-04 | RNA probe_03, LwaCas13a-crRNA_03 and |
| | LwaCas13a-crRNA_04 |
| G-04 | RNA probe_04, LwaCas13a-crRNA_04 and |
| | LwaCas13a-crRNA_05 |
| H-04 | RNA probe_05, LwaCas13a-crRNA_05 and |
| | LwaCas13a-crRNA_06 |
| I-04 | RNA probe_06, LwaCas13a-crRNA_06 and |
| | LwaCas13a-crRNA_07 |
| D-05 | RNA probe_07, LwaCas13a-crRNA_07 and |
| | LwaCas13a-crRNA_08 |
| E-05 | RNA probe_08, LwaCas13a-crRNA_08 and |
| | LwaCas13a-crRNA_09 |
| F-05 | RNA probe_09, LwaCas13a-crRNA_09 and |
| | LwaCas13a-crRNA_10 |
| G-05 | RNA probe_10, LwaCas13a-crRNA_10 and |
| | LwaCas13a-crRNA_11 |
| H-05 | RNA probe_11, LwaCas13a-crRNA_11 and |
| | LwaCas13a-crRNA_12 |
| I-05 | RNA probe_12, LwaCas13a-crRNA_12 and |
| | LwaCas13a-crRNA_13 |
| D-06 | RNA probe_13, LwaCas13a-crRNA_13 and |
| | LwaCas13a-crRNA_14 |
| E-06 | RNA probe_14, LwaCas13a-crRNA_14 and |
| | LwaCas13a-crRNA_15 |
| F-06 | RNA probe_15, LwaCas13a-crRNA_15 and |
| | LwaCas13a-crRNA_16 |
| G-06 | RNA probe_16, LwaCas13a-crRNA_16 and |
| | LwaCas13a-crRNA_17 |
| H-06 | RNA probe_17, LwaCas13a-crRNA_17 and |
| | LwaCas13a-crRNA_18 |
| I-06 | RNA probe_18, LwaCas13a-crRNA_18 and |
| | LwaCas13a-crRNA_19 |
| D-07 | RNA probe_19, LwaCas13a-crRNA_19 and |
| | LwaCas13a-crRNA_20 |
| E-07 | RNA probe_20, LwaCas13a-crRNA_20 and |
| | LwaCas13a-crRNA_21 |
| F-07 | RNA probe_21, LwaCas13a-crRNA_21 and |
| | LwaCas13a-crRNA_22 |
| G-07 | RNA probe_22, LwaCas13a-crRNA_22 and |
| | LwaCas13a-crRNA_23 |
| H-07 | RNA probe_23, LwaCas13a-crRNA_23 and |
| | LwaCas13a-crRNA_24 |
| I-07 | RNA probe_24, LwaCas13a-crRNA_24 and |
| | LwaCas13a-crRNA_25 |
| D-08 | RNA probe_25, LwaCas13a-crRNA_25 and |
| | LwaCas13a-crRNA_26 |
| E-08 | RNA probe_26, LwaCas13a-crRNA_26 and |
| | LwaCas13a-crRNA_27 |
| F-08 | RNA probe_27, LwaCas13a-crRNA_27 and |
| | LwaCas13a-crRNA_28 |
| G-08 | RNA probe_28, LwaCas13a-crRNA_28 and |
| | LwaCas13a-crRNA_29 |
| H-08 | RNA probe_29, LwaCas13a-crRNA_29 and |
| | LwaCas13a-crRNA_30 |
| I-08 | RNA probe_30, LwaCas13a-crRNA_30 and |
| | LwaCas13a-crRNA_31 |
| D-09 | RNA probe_31, LwaCas13a-crRNA_31 and |
| | LwaCas13a-crRNA_32 |
| E-09 | RNA probe_32, LwaCas13a-crRNA_32 and |
| | LwaCas13a-crRNA_33 |

TABLE 6

| | | Eₕ | | |
|---|---|---|---|---|
| | ΔE | (target + | Eᵣₙₐ₁ | Eᵣₙₐ₂ |
| Group | (kcal/mol) | probe) | (target) | (probe) |
| Target_01 + probe_01 | −14.06 | −19.60 | −0.74 | −4.80 |
| Target_02 + probe_02 | −11.70 | −25.30 | −6.20 | −7.40 |
| Target_03 + probe_03 | −9.60 | −18.50 | −5.70 | −3.20 |
| Target_04 + probe_04 | −8.50 | −22.40 | −8.00 | −5.90 |
| Target_05 + probe_05 | −7.30 | −20.00 | −8.60 | −4.10 |
| Target_06 + probe_06 | −9.20 | −34.40 | −15.80 | −9.40 |
| Target_07 + probe_07 | −16.20 | −37.10 | −15.50 | −5.40 |
| Target_08 + probe_08 | −12.00 | −27.60 | −4.90 | −10.70 |
| Target_09 + probe_09 | −10.30 | −28.90 | −9.40 | −9.20 |
| Target_10 + probe_10 | −14.40 | −31.20 | −7.60 | −9.20 |
| Target_11 + probe_11 | −5.20 | −14.60 | −5.80 | −3.60 |
| Target_12 + probe_12 | −8.40 | −24.40 | −10.20 | −5.80 |
| Target_13 + probe_13 | −5.00 | −38.00 | −18.60 | −14.40 |
| Target_14 + probe_14 | −9.20 | −22.50 | −8.00 | −5.30 |
| Target_15 + probe_15 | −15.20 | −42.80 | −15.10 | −12.50 |
| Target_16 + probe_16 | −13.20 | −31.40 | −9.40 | −8.80 |
| Target_17 + probe_17 | −8.70 | −32.10 | −14.10 | −9.30 |
| Target_18 + probe_18 | −6.90 | −17.40 | −4.10 | −6.40 |
| Target_19 + probe_19 | −5.90 | −23.80 | −9.40 | −8.50 |
| Target_20 + probe_20 | −7.50 | −20.50 | −6.10 | −6.90 |
| Target_21 + probe_21 | −1.40 | −34.50 | −17.60 | −15.50 |
| Target_22 + probe_22 | −3.60 | −18.00 | −5.40 | −9.00 |
| Target_23 + probe_23 | −12.70 | −34.60 | −11.50 | −10.40 |
| Target_24 + probe_24 | −10.90 | −29.60 | −8.80 | −9.90 |
| Target_25 + probe_25 | −6.50 | −21.30 | −9.80 | −5.00 |
| Target_26 + probe_26 | −8.60 | −30.50 | −11.30 | −10.60 |
| Target_27 + probe_27 | −3.70 | −31.60 | −14.70 | −13.20 |
| Target_28 + probe_28 | −10.30 | −26.10 | −9.60 | −6.20 |
| Target_29 + probe_29 | −8.00 | −19.80 | −5.40 | −6.40 |
| Target_30 + probe_30 | −6.10 | −21.70 | −7.00 | −8.60 |
| Target_31 + probe_31 | −2.90 | −25.20 | −16.40 | −5.90 |
| Target_32 + probe_32 | −8.20 | −26.00 | −13.80 | −4.00 |

TABLE 7

| | ΔE | Eₕ (crRNA + | Eᵣₙₐ₁ | Eᵣₙₐ₂ |
|---|---|---|---|---|
| Group | (kcal/mol) | protospacer) | (crRNA) | (protospacer) |
| crRNA_01 + | −10.40 | −17.10 | −6.70 | 0.00 |
| protospacer 01 | | | | |
| crRNA_02 + | −5.40 | −19.80 | −11.50 | −2.90 |
| protospacer 02 | | | | |
| crRNA_03 + | −5.70 | −21.50 | −10.50 | −5.30 |
| protospacer 03 | | | | |
| crRNA_04 + | −8.50 | −17.00 | −8.50 | −0.00 |
| protospacer 04 | | | | |
| crRNA_05 + | −9.40 | −16.50 | −7.00 | −0.10 |
| protospacer 05 | | | | |
| crRNA_06 + | −10.40 | −19.40 | −8.50 | −0.50 |
| protospacer 06 | | | | |
| crRNA_07 + | −10.30 | −19.50 | −8.70 | −0.50 |
| protospacer 07 | | | | |
| crRNA_08 + | −14.40 | −26.20 | −9.00 | −2.80 |
| protospacer 08 | | | | |
| crRNA_09 + | −12.00 | −22.80 | −9.20 | −1.60 |
| protospacer 09 | | | | |
| crRNA_10 + | −12.30 | −26.20 | −9.40 | −4.50 |
| protospacer 10 | | | | |
| crRNA_11 + | −3.90 | −17.10 | −8.70 | −4.50 |
| protospacer 11 | | | | |
| crRNA_12 + | −5.80 | −20.10 | −6.90 | −7.40 |
| protospacer 12 | | | | |
| crRNA_13 + | −8.30 | −14.40 | −6.10 | 0.00 |
| protospacer 13 | | | | |
| crRNA_14 + | −8.90 | −20.50 | −9.20 | −2.40 |
| protospacer 14 | | | | |
| crRNA_15 + | −8.50 | −24.00 | −12.00 | −3.50 |
| protospacer 15 | | | | |
| crRNA_16 + | −5.60 | −15.20 | −7.70 | −1.90 |
| protospacer 16 | | | | |
| crRNA_17 + | −9.80 | −18.60 | −7.50 | −1.30 |
| protospacer 17 | | | | |
| crRNA_18 + | −6.60 | −19.30 | −9.60 | −3.10 |
| protospacer 18 | | | | |
| crRNA_19 + | −8.30 | −18.90 | −9.40 | −1.20 |
| protospacer 19 | | | | |
| crRNA_20 + | −5.50 | −17.60 | −10.70 | −1.40 |
| protospacer 20 | | | | |
| crRNA_21 + | −6.90 | −16.10 | −7.00 | −2.20 |
| protospacer 21 | | | | |
| crRNA_22 + | −13.50 | −21.80 | −6.60 | −1.70 |
| protospacer 22 | | | | |
| crRNA_23 + | −10.30 | −22.70 | −8.20 | −4.20 |
| protospacer 23 | | | | |
| crRNA_24 + | −10.50 | −19.90 | −9.00 | −0.40 |
| protospacer 24 | | | | |
| crRNA_25 + | −2.60 | −12.60 | −7.70 | −2.30 |
| protospacer 25 | | | | |
| crRNA_26 + | −10.40 | −18.90 | −6.80 | −1.70 |
| protospacer 26 | | | | |
| crRNA_27 + | −3.90 | −14.80 | −8.50 | −2.40 |
| protospacer 27 | | | | |
| crRNA_28 + | −6.90 | −20.70 | −9.40 | −4.40 |
| protospacer 28 | | | | |
| crRNA_29 + | −9.90 | −22.70 | −9.60 | −3.20 |
| protospacer 29 | | | | |
| crRNA_30 + | −3.40 | −22.30 | −12.70 | −6.20 |
| protospacer 30 | | | | |
| crRNA_31 + | −7.10 | −23.40 | −13.10 | −3.20 |
| protospacer 31 | | | | |
| crRNA_32 + | −7.80 | −20.10 | −8.20 | −4.10 |
| protospacer 32 | | | | |
| crRNA_33 + | −9.00 | −19.30 | −10.30 | 0.00 |
| protospacer 33 | | | | |

TABLE 8

| | ΔE | Eₕ (crRNA + | Eᵣₙₐ₁ | Eᵣₙₐ₂ |
|---|---|---|---|---|
| Group | (kcal/mol) | probe) | (crRNA) | (probe) |
| crRNA_01 + probe_01 | −9.70 | −21.20 | −6.70 | −4.80 |
| crRNA_02 + probe_02 | −2.80 | −21.70 | −11.50 | −7.40 |
| crRNA_03 + probe_03 | −3.90 | −17.60 | −10.50 | −3.20 |
| crRNA_04 + probe_04 | −4.00 | −18.40 | −8.50 | −5.90 |
| crRNA_05 + probe_05 | −4.10 | −15.20 | −7.00 | −4.10 |
| crRNA_06 + probe_06 | −4.20 | −22.10 | −8.50 | −9.40 |
| crRNA_07 + probe_07 | −5.20 | −19.30 | −8.70 | −5.40 |
| crRNA_08 + probe_08 | −1.30 | −21.00 | −9.00 | −10.70 |
| crRNA_09 + probe_09 | −5.00 | −23.40 | −9.20 | −9.20 |
| crRNA_10 + probe_10 | −2.30 | −20.90 | −9.40 | −9.20 |
| crRNA_11 + probe_11 | −3.80 | −16.10 | −8.70 | −3.60 |
| crRNA_12 + probe_12 | −7.80 | −20.50 | −6.90 | −5.80 |
| crRNA_13 + probe_13 | −7.20 | −27.70 | −6.10 | −14.40 |
| crRNA_14 + probe_14 | −1.30 | −15.80 | −9.20 | −5.30 |
| crRNA_15 + probe_15 | −1.60 | −26.10 | −12.00 | −12.50 |
| crRNA_16 + probe_16 | −10.10 | −26.60 | −7.70 | −8.80 |
| crRNA_17 + probe_17 | −4.10 | −20.90 | −7.50 | −9.30 |
| crRNA_18 + probe_18 | −7.80 | −23.80 | −9.60 | −6.40 |
| crRNA_19 + probe_19 | −7.60 | −25.50 | −9.40 | −8.50 |
| crRNA_20 + probe_20 | −6.90 | −24.50 | −10.70 | −6.90 |
| crRNA_21 + probe_21 | −3.10 | −25.60 | −7.00 | −15.50 |
| crRNA_22 + probe_22 | −3.90 | −19.50 | −6.60 | −9.00 |
| crRNA_23 + probe_23 | −1.10 | −19.70 | −8.20 | −10.40 |
| crRNA_24 + probe_24 | −6.20 | −25.10 | −9.00 | −9.90 |
| crRNA_25 + probe_25 | −4.10 | −16.80 | −7.70 | −5.00 |
| crRNA_26 + probe_26 | −8.30 | −25.70 | −6.80 | −10.60 |
| crRNA_27 + probe_27 | −4.30 | −26.00 | −8.50 | −13.20 |
| crRNA_28 + probe_28 | −6.60 | −22.20 | −9.40 | −6.20 |
| crRNA_29 + probe_29 | −10.90 | −26.90 | −9.60 | −6.40 |
| crRNA_30 + probe_30 | −2.50 | −23.80 | −12.70 | −8.60 |
| crRNA_31 + probe_31 | −4.00 | −23.00 | −13.10 | −5.90 |
| crRNA_32 + probe_32 | −6.60 | −18.80 | −8.20 | −4.00 |

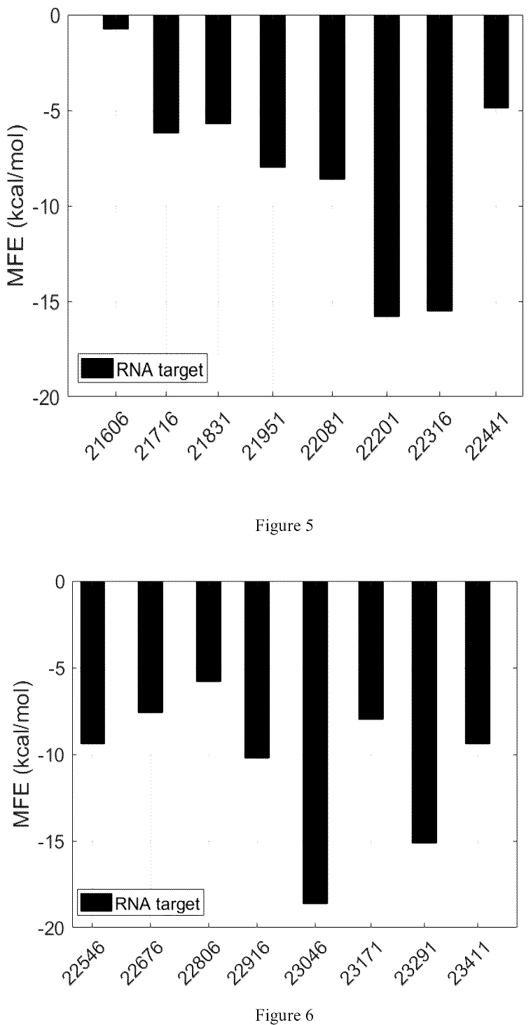

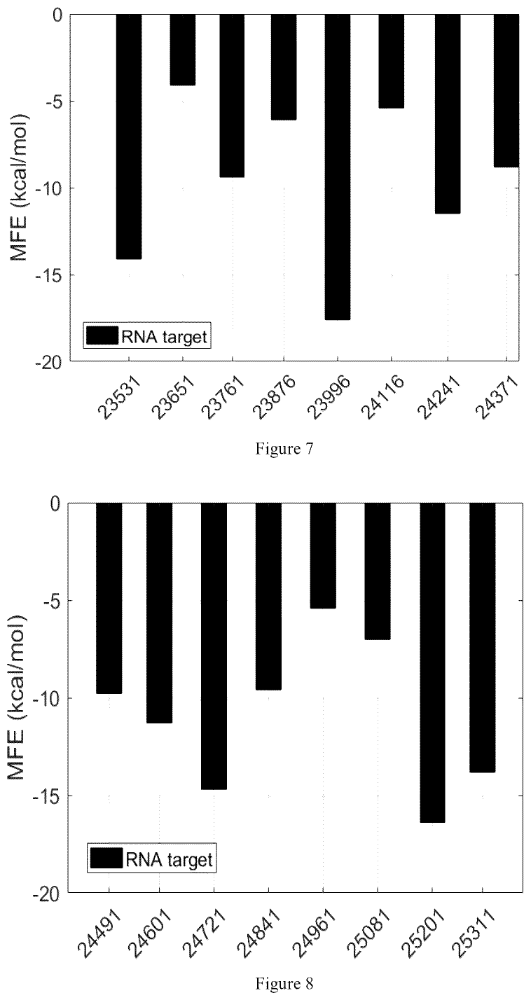

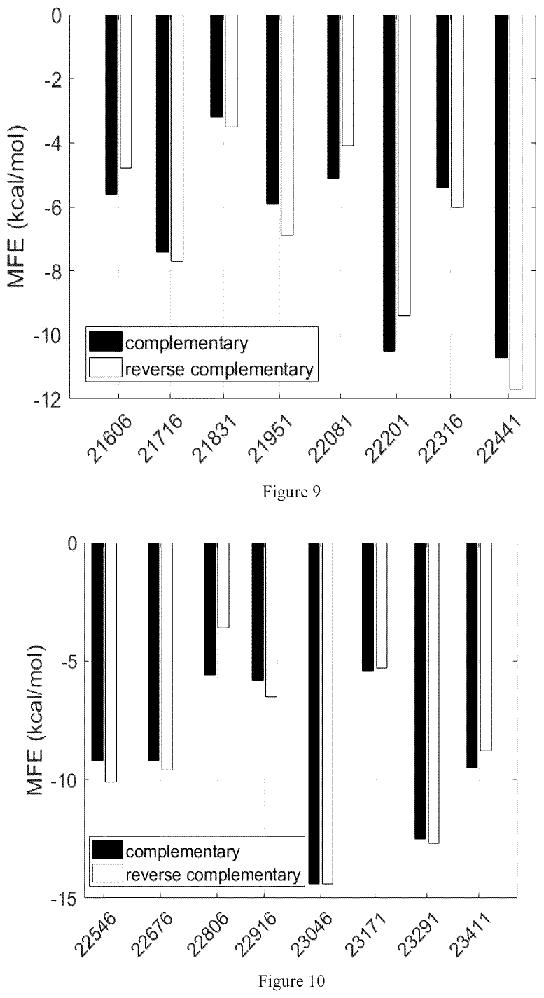

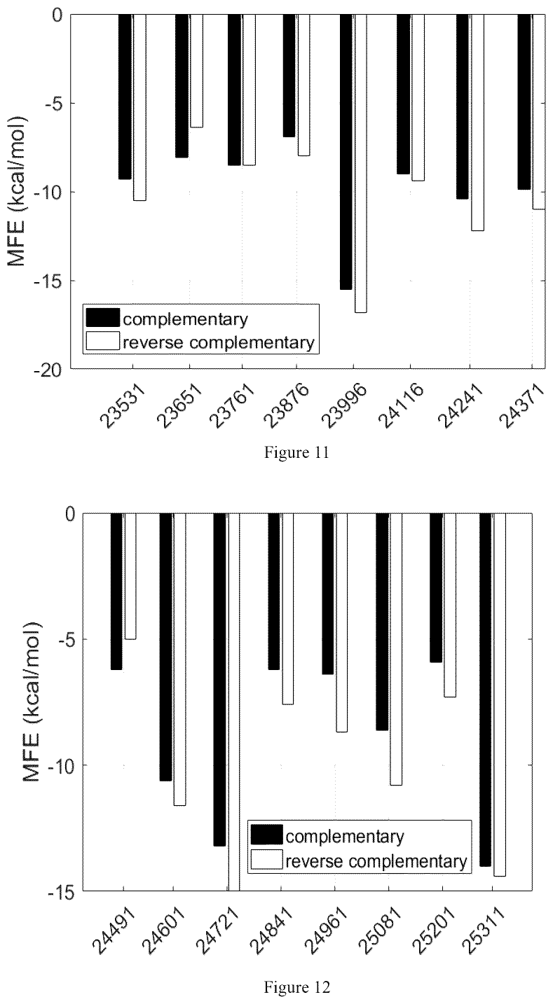

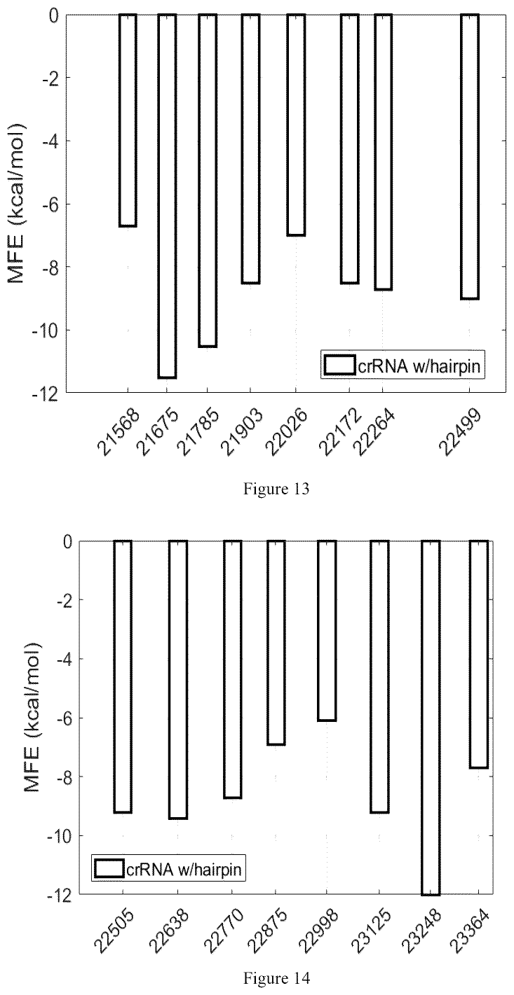

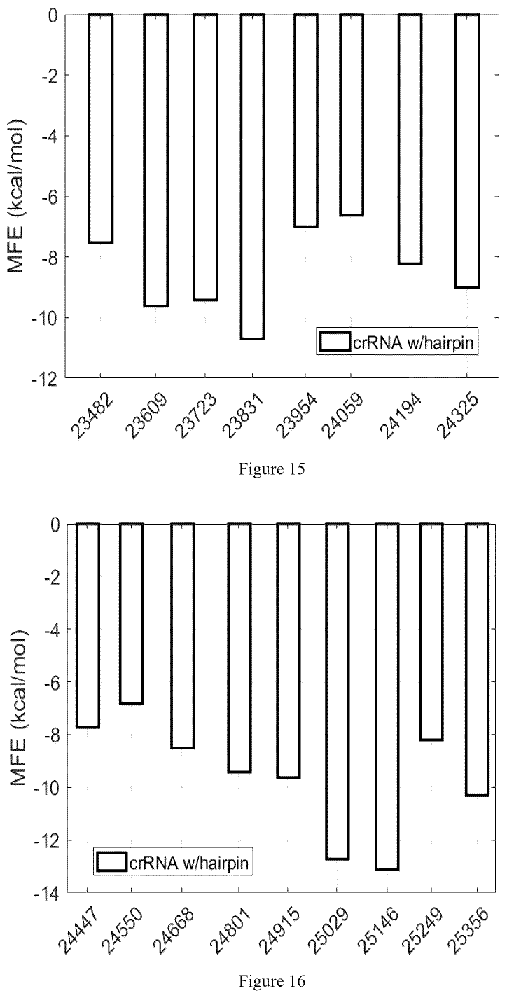

The calculation results are shown in Tables 1-4, Tables 6-9 and FIGS. 5-17. Specifically, the MFE of 32 cleaved spike RNA target is listed in Table 1, the MFE of 32 protospacer on the spike RNA sequence is listed in Table 2, and the MFE of 32 probe sequence is listed in Table 3. The less negative the MFE, the higher the RNA reactivity. So, the probe with less negative MPE was selected for energy calculation and sensing of the RNA target. The MFE of 33 crRNA sequence is listed in Table 4. The average change of MFE in the three scenarios, i.e., (1) target-probe (see FIG. 6), (2) crRNA-protospacer (see FIG. 7), and (3) nonspecific crRNA-probe (see FIG. 8) are −8.79 kcal/mol, −8.25 kcal/mol, and −5.07 kcal/mol, respectively. Target-probe sequence and crRNA-protospacer sequence with the absolute value of ΔE greater than 8 kcal/mol were selected, and the absolute value of ΔE of non-specific hybridization was required to be less than 5 kcal/mol. The minimum free energy of spike RNA target sequence are plot in FIGS. 5-8, from which the minimum free energy of the RNA target sequence can be seen. The minimum free energy of complementary and reverse complementary probes is plotted in FIGS. 9-12, from which the minimum free energy of the complementary and reverse complementary RNA of spike target sequence can be seen. The minimum free energy of crRNA is plotted in FIGS. 13-16, from which the minimum free energy of the crRNA can be seen. Through the screening conditions of ΔE and the data in Tables 6-8, the optimal combination of MFE was obtained and listed in Table 9.

TABLE 9

| | ΔE | | ΔE | Optimal |
|---|---|---|---|---|
| | target + | ΔEcrRNA + | crRNA + | combination |
| Cell ID on | probe | protospacer | probe | of MFE |
| microarray | (kcal/mol) | (kcal/mol) | (kcal/mol) | (Yes/No) |
| D-04 | −14.06 | −10.40 | −9.70 | No |
| E-04 | −11.70 | −5.40 | −2.80 | No |
| F-04 | −9.60 | −5.70 | −3.90 | No |
| G-04 | −8.50 | −8.50 | −4.00 | Yes |
| H-04 | −7.30 | −9.40 | −4.10 | No |
| I-04 | −9.20 | −10.40 | −4.20 | Yes |
| D-05 | −16.20 | −10.30 | −5.20 | No |
| E-05 | −12.00 | −14.40 | −1.30 | Yes |
| F-05 | −10.30 | −12.00 | −5.00 | Yes |
| G-05 | −14.40 | −12.30 | −2.30 | Yes |
| H-05 | −5.20 | −3.90 | −3.80 | No |
| I-05 | −8.40 | −5.80 | −7.80 | No |
| D-06 | −5.00 | −8.30 | −7.20 | No |
| E-06 | −9.20 | −8.90 | −1.30 | Yes |
| F-06 | −15.20 | −8.50 | −1.60 | Yes |
| G-06 | −13.20 | −5.60 | −10.10 | No |
| H-06 | −8.70 | −9.80 | −4.10 | Yes |
| I-06 | −6.90 | −6.60 | −7.80 | No |
| D-07 | −5.90 | −8.30 | −7.60 | No |
| E-07 | −7.50 | −5.50 | −6.90 | No |
| F-07 | −1.40 | −6.90 | −3.10 | No |
| G-07 | −3.60 | −13.50 | −3.90 | No |
| H-07 | −12.70 | −10.30 | −1.10 | Yes |
| I-07 | −10.90 | −10.50 | −6.20 | No |
| D-08 | −6.50 | −2.60 | −4.10 | No |
| E-08 | −8.60 | −10.40 | −8.30 | No |
| F-08 | −3.70 | −3.90 | −4.30 | No |
| G-08 | −10.30 | −6.90 | −6.60 | No |
| H-08 | −8.00 | −9.90 | −10.90 | No |
| I-08 | −6.10 | −3.40 | −2.50 | No |
| D-09 | −2.90 | −7.10 | −4.00 | No |
| E-09 | −8.20 | −7.80 | −6.60 | No |

As can be seen from Table 9 that the flow cells numbered G04, I04, E05, G05, E06, F06, H06 and H07 obtained through screening in this example have better effects.

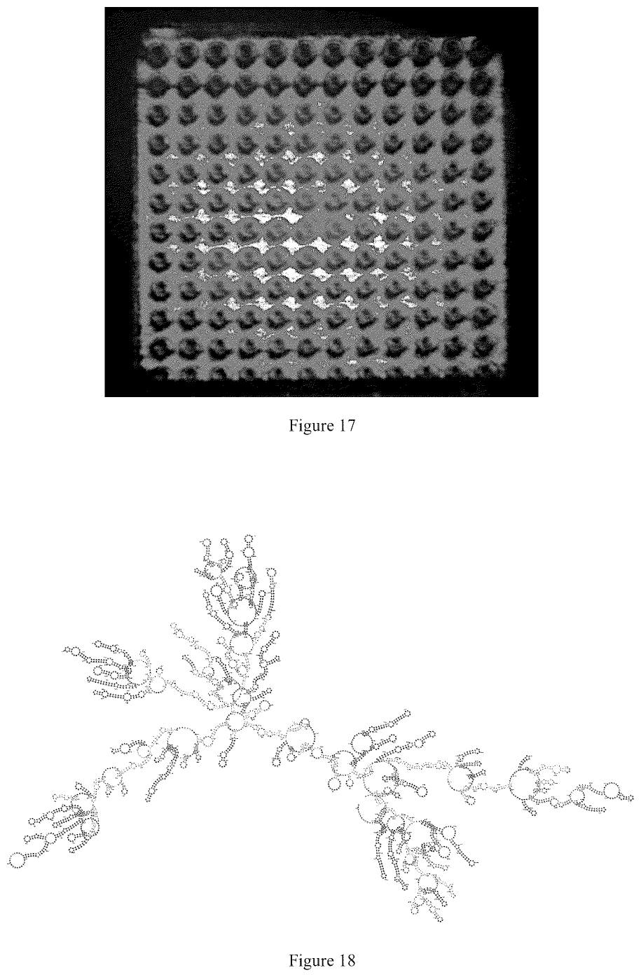

FIG. 17 shows the image of the 12× 12 plasmonic microarray for sequential nucleic acid test of the SARS-COV-2 spike fragments by hybridization. Multiple SARS-COV-2 spike RNA gene sequence fragments were detected by CRISPR-Cas13a cleavage and the change in MFE between reactants was computationally evaluated. Furthermore, for the detection sensitivity on SARS-COV-2 spike fragment, the scheme of the present disclosure is 8%-25% higher than that of the fluorescent quantitative polymerase chain reaction, and has a great application prospect.

FIG. 18 shows the secondary RNA structure of the complete spike gene with total of 3821 ribonucleic acid bases (21563 to 25384 bp) computed by the minimum free energy approach. Annotations: A, U, C, and G represent adenine, uracil, cytosine and guanine, respectively. The color of each annotation represents the base-pairing probability, dark colors indicate high likelihood of forming A-U or C-G base-pairs and light colors indicates the opposite. The accompanied number indicates position (1 to 3821) of the spike RNA sequence. The secondary structure was obtained by inputting the complete spike RNA gene sequence of 3821 ribonucleic acid bases and all calculations are done via the RNAfold module of ViennaRNA Package 2.0.

### Example 3 SARS-COV-2 Nucleic Acid Detection

The SARS-COV-2 RT-PCR nucleic acid detection kit (#MFG030015) produced by BGIEurope A/S was used as the reference sample. The positive sample provided in the kit was diluted to obtain a positive sample target solution with a Ct value of 25 and a target nucleic acid concentration of approximately 1 pM.

The operation steps were the same as in Example 2. The solution and 1 ng/ml titanium nitride nanocube suspension (RNA probes and LwaCas13a-crRNA complex pre-functionalized titanium nitride nanocubes in G-04, I-04, E-05, F-05, G-05, E-06, F-06, H-06 and H-07 flow cells shown in Table 5, respectively) were mixed, and then the mixed solution was injected into the biochip at a flow rate of 100 μL per minute through a micropump. Hybridizations of nucleic acid target fragments and probes were measured through cleavage by the CRISPR-Cas13a enzyme and the thermoplasmonic resonance effect of titanium nitride nanocubes. Moreover, the phase data of the surrounding non-functional flow cells were collected for comparison.

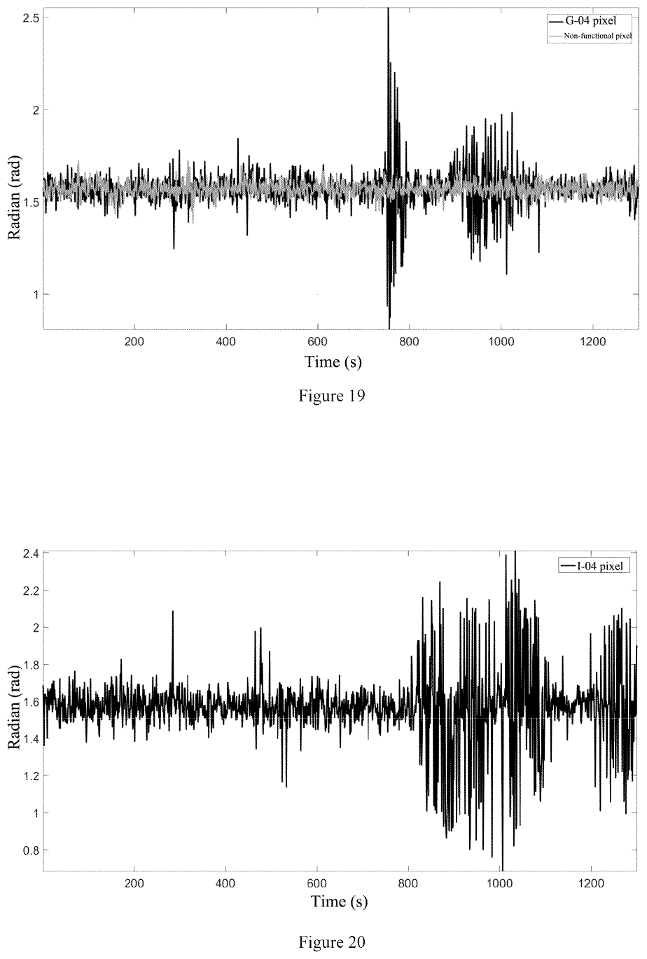

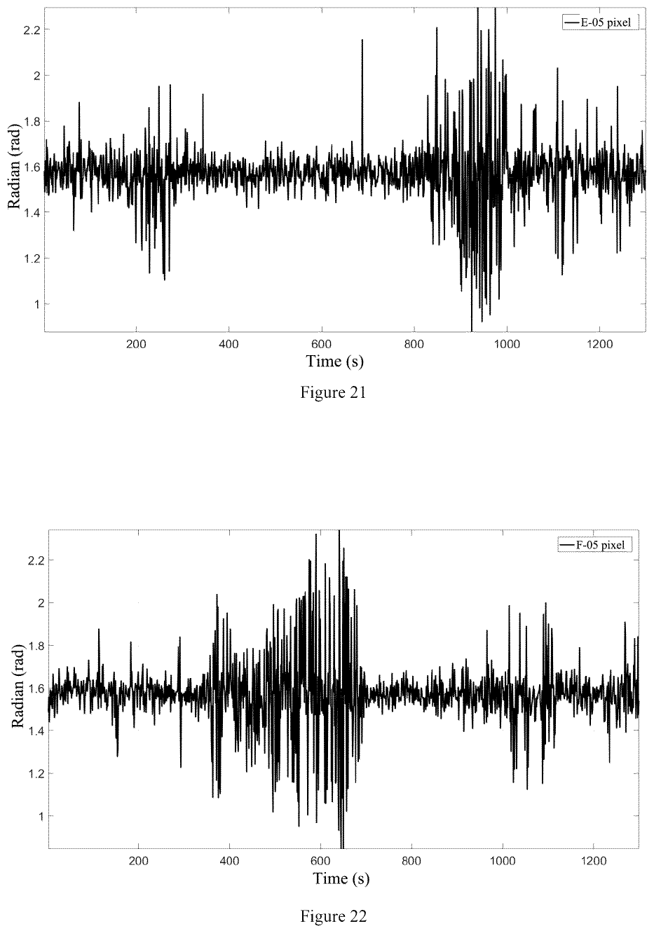

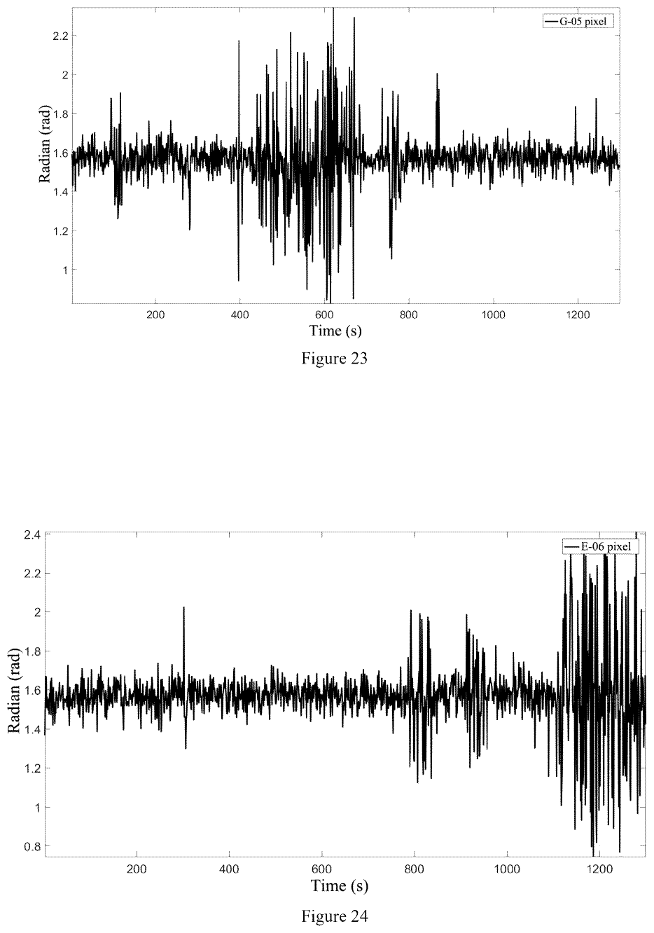

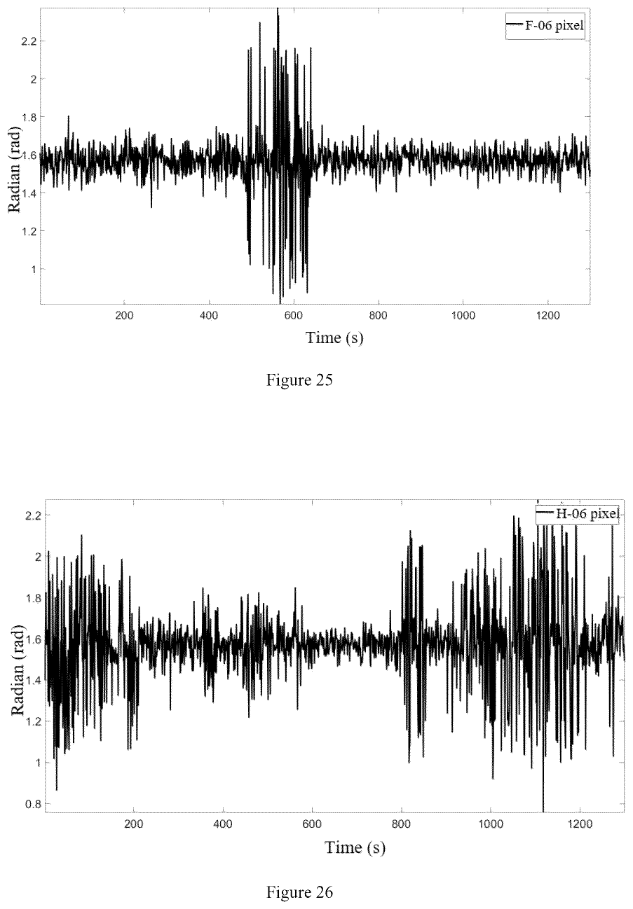

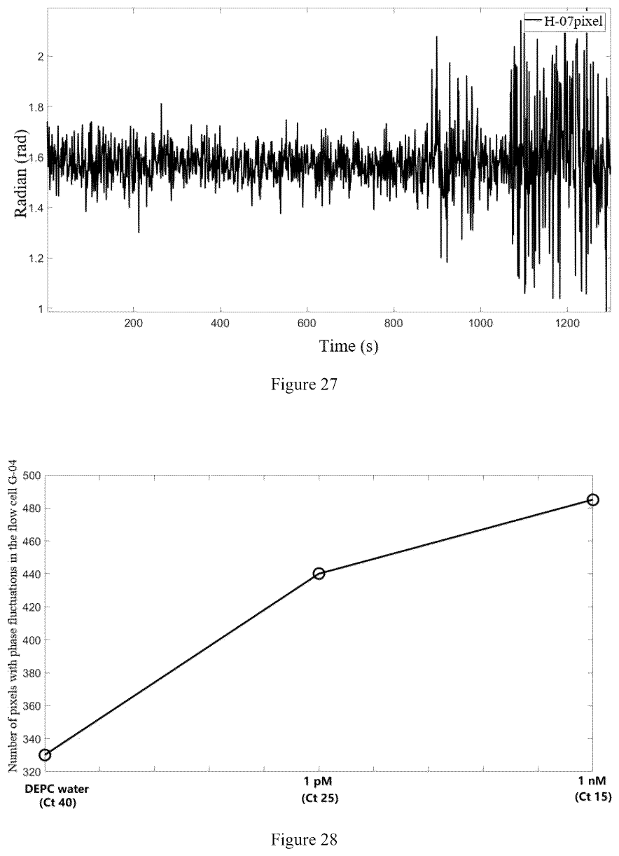

The results are shown in FIGS. 19-28. FIG. 19 demonstrates that obvious pixel phase fluctuations can be found in the G-04 flow cell, while the pixel phase change of the non-functionalized flow cell A-01 is not obvious. FIGS. 20-27 show the fluctuation data for pixels of flow cells I-04, E-05, F-05, G-05, E-06, F-06, H-06 and H-07, respectively.

The number of pixels with phase fluctuations in the flow cell was screened and counted as an indicator for quantitative measurement of nucleic acid concentration. Nuclease-free water (a Ct value of approximately 40), 1 pM positive nucleic acid sample (a Ct value of approximately 25), and 1 nM positive nucleic acid sample (a Ct value of approximately 15) were sequentially injected into the chip for measurement by using the method described above.

The G-04 flow cell data of these three sets of concentrations are shown in FIG. 28, from which a clear linear trend can be observed, demonstrating that the present disclosure can be used to quantitatively measure the concentration of injected nucleic acids.

The embodiments of the present disclosure have been described in detail above in conjunction with the accompanying drawings, but the present disclosure is not limited to the above-mentioned embodiments. Within the scope of knowledge of those skilled in the art, various modifications can be made without departing from the spirit of the present disclosure. In addition, the embodiments of the present disclosures and the features in the embodiments can be combined with each other if there is no conflict.

1. A CRISPR-Cas13 system for detecting SARS-COV-2, comprising:
a Cas13a protein and crRNA; or
a complex formed by Cas13a protein and crRNA,
wherein the crRNA comprises a first guide RNA and a second guide RNA;
wherein the first guide RNA and the second guide RNA comprise at least one sequence selected from the group consisting of SEQ ID NOs: 1 to 33, respectively.

2. The CRISPR-Cas13 system according to claim 1, wherein the crRNA is a guide sequence designed for a SARS-COV-2 target gene.

3. The CRISPR-Cas13 system according to claim 2, wherein the SARS-COV-2 target sequence comprises at least one sequence selected from the group consisting of SEQ ID NOs: 34 to 65.

4. The CRISPR-Cas13 system according to claim 1, further comprising a probe sequence, wherein the probe sequence comprises at least one sequence selected from the group consisting of SEQ ID NOs: 66 to 97.

5. The CRISPR-Cas13 system according to claim 1, wherein the first guide RNA and the second guide RNA comprise different sequences; and the first guide RNA sequence and the second guide RNA sequence are used to cleave the same segment of the SARS-COV-2 target sequence.

6. The CRISPR-Cas13 system according to claim 1, wherein the Cas13a protein is LwCas13a protein.

7. A kit for detecting SARS-COV-2, comprising the CRISPR-Cas13 system according to claim 1.

8. The kit according to claim 7, further comprising an RNA extraction kit, a LSPR biosensor, and a plasmonic waveguide microarray chip.

9-17. (canceled)

18. A method for detecting or aiding in the detection of SARS-COV-2 nucleic acid, comprising:
(1) extracting nucleic acid from a sample to be tested;
(2) adding the CRISPR-Cas13 system of claim 1, a probe, titanium nitride nanocubes and the nucleic acid from the sample to be tested to a plasma waveguide microarray chip and leaving it to stand for observation; and
(3) using a LSPR biosensor for signal detection, wherein detection of a signal indicates that the sample to be tested contains the SARS-COV-2 or is a candidate that contains the SARS-COV-2 and lack of a signal indicates that the sample to be tested does not contain the SARS-COV-2 or is a candidate that does not contain the SARS-COV-2.

19. The CRISPR-Cas13 system of claim 2, wherein the SARS-COV-2 target gene encodes a spike protein.


SEQUENCE LISTING (130 sequences)

SEQ ID NO: 1 - RNA, 57 nt, synthetic construct
  source 1..57: mol_type = other RNA
acaaaaagaacaaaataacggtgatcagacaaaatcaggggaagctataaccccacc

SEQ ID NO: 2 - RNA, 57 nt, synthetic construct
  source 1..57: mol_type = other RNA
tgggactgtttcaaaagtctaggagtcaacaaaatcaggggaagctataaccccacc

SEQ ID NO: 3 - RNA, 57 nt, synthetic construct
  source 1..57: mol_type = other RNA
ccatgattctccaaactattgggacaggacaaaatcaggggaagctataaccccacc

SEQ ID NO: 4 - RNA, 57 nt, synthetic construct
  source 1..57: mol_type = other RNA
gggtcagggatgaataacaattattgcgacaaaatcaggggaagctataaccccacc

SEQ ID NO: 5 - RNA, 57 nt, synthetic construct
  source 1..57: mol_type = other RNA
cactcaagtctcaaataagatcacgcttacaaaatcaggggaagctataaccccacc

SEQ ID NO: 6 - RNA, 57 nt, synthetic construct
  source 1..57: mol_type = other RNA
ataagattcgtgtgcggataattaaatcacaaaatcaggggaagctataaccccacc

SEQ ID NO: 7 - RNA, 57 nt, synthetic construct
  source 1..57: mol_type = other RNA
gtagtgatccaaagtttgaaatgaacgaacaaaatcaggggaagctataaccccacc

SEQ ID NO: 8 - RNA, 57 nt, synthetic construct
  source 1..57: mol_type = other RNA
ccttggtaatgtctacgacatctgacacgcaaaatcaggggaagctataaccccacc

SEQ ID NO: 9 - RNA, 57 nt, synthetic construct
  source 1..57: mol_type = other RNA
tgaagattgaaatctcaggttggttgtctcaaaatcaggggaagctataaccccacc

SEQ ID NO: 10 - RNA, 57 nt, synthetic construct
  source 1..57: mol_type = other RNA
cgttgacacaacgactaataagacaggatcaaaatcaggggaagctataaccccacc

SEQ ID NO: 11 - RNA, 57 nt, synthetic construct
  source 1..57: mol_type = other RNA
ctccactacttcagtctgtttagcgaggtcaaaatcaggggaagctataaccccacc

SEQ ID NO: 12 - RNA, 57 nt, synthetic construct
  source 1..57: mol_type = other RNA
gattgttagaactaagattccaaccaccacaaaatcaggggaagctataaccccacc

SEQ ID NO: 13 - RNA, 57 nt, synthetic construct
  source 1..57: mol_type = other RNA
gaacattaccacaacttccaaaattaacacaaaatcaggggaagctataaccccacc

SEQ ID NO: 14 - RNA, 57 nt, synthetic construct
  source 1..57: mol_type = other RNA
tcgttgacaaacacctggatttttcagatcaaaatcaggggaagctataaccccacc

SEQ ID NO: 15 - RNA, 57 nt, synthetic construct
  source 1..57: mol_type = other RNA
ggttgttaaaccgtctctgtaacgactgtcaaaatcaggggaagctataaccccacc

SEQ ID NO: 16 - RNA, 57 nt, synthetic construct
  source 1..57: mol_type = other RNA
cttgtttatgaagattggtccaacgacaacaaaatcaggggaagctataaccccacc

SEQ ID NO: 17 - RNA, 57 nt, synthetic construct
  source 1..57: mol_type = other RNA
attacaaaaagtttgtgcacgtccgacaacaaaatcaggggaagctataaccccacc

SEQ ID NO: 18 - RNA, 57 nt, synthetic construct
  source 1..57: mol_type = other RNA
gcccgtgcatcacatcgatcagttaggtacaaaatcaggggaagctataaccccacc

SEQ ID NO: 19 - RNA, 57 nt, synthetic construct
  source 1..57: mol_type = other RNA
tcacaatggtgtctttaagatggtcacagcaaaatcaggggaagctataaccccacc

SEQ ID NO: 20 - RNA, 57 nt, synthetic construct
  source 1..57: mol_type = other RNA
ccgtcaaaaacatgtgttaatttggcacgcaaaatcaggggaagctataaccccacc

SEQ ID NO: 21 - RNA, 57 nt, synthetic construct
  source 1..57: mol_type = other RNA
ccaccaaaattaaaaagtgtttataatggcaaaatcaggggaagctataaccccacc

SEQ ID NO: 22 - RNA, 57 nt, synthetic construct
  source 1..57: mol_type = other RNA
ttataccactaacggaaccactataacgacaaaatcaggggaagctataaccccacc

SEQ ID NO: 23 - RNA, 57 nt, synthetic construct
  source 1..57: mol_type = other RNA
aatcgcccatgttagtgaagaccaacctgcaaaatcaggggaagctataaccccacc

SEQ ID NO: 24 - RNA, 57 nt, synthetic construct
  source 1..57: mol_type = other RNA
taactaacggttggttaaattatcacgatcaaaatcaggggaagctataaccccacc

SEQ ID NO: 25 - RNA, 57 nt, synthetic construct
  source 1..57: mol_type = other RNA
aacaatttgttgaatcgaggttaaaaccacaaaatcaggggaagctataaccccacc

SEQ ID NO: 26 - RNA, 57 nt, synthetic construct
  source 1..57: mol_type = other RNA
tctgaagtttcaaacgtctgtatacactgcaaaatcaggggaagctataaccccacc

SEQ ID NO: 27 - RNA, 57 nt, synthetic construct
  source 1..57: mol_type = other RNA
gttagtttttctcaactaaaaacacctttcaaaatcaggggaagctataaccccacc

SEQ ID NO: 28 - RNA, 57 nt, synthetic construct
  source 1..57: mol_type = other RNA
ggtaaacagtactaccttttcgtgtgaaacaaaatcaggggaagctataaccccacc

SEQ ID NO: 29 - RNA, 57 nt, synthetic construct
  source 1..57: mol_type = other RNA
tgttgtgtaaacacagaccattgacactacaaaatcaggggaagctataaccccacc

SEQ ID NO: 30 - RNA, 57 nt, synthetic construct
  source 1..57: mol_type = other RNA
aattcttagtatgtagtggtctacaactacaaaatcaggggaagctataaccccacc

SEQ ID NO: 31 - RNA, 57 nt, synthetic construct
  source 1..57: mol_type = other RNA
ttagagagtagctagaggttcttgaacctcaaaatcaggggaagctataaccccacc

SEQ ID NO: 32 - RNA, 57 nt, synthetic construct
  source 1..57: mol_type = other RNA
ccactgttaatacgaaacgacatactggtcaaaatcaggggaagctataaccccacc

SEQ ID NO: 33 - RNA, 57 nt, synthetic construct
  source 1..57: mol_type = other RNA
agtttcctcagtttaatgtaatgtgtattcaaaatcaggggaagctataaccccacc

SEQ ID NO: 34 - RNA, 60 nt, SARS-CoV-2
  source 1..60: mol_type = other RNA
gtgttaatcttacaaccagaactcaattaccccctgcatacactaattctttcacacgtg

SEQ ID NO: 35 - RNA, 60 nt, SARS-CoV-2
  source 1..60: mol_type = other RNA
caggacttgttcttacctttcttttccaatgttacttggttccatgctatacatgtctct

SEQ ID NO: 36 - RNA, 60 nt, SARS-CoV-2
  source 1..60: mol_type = other RNA
tttattttgcttccactgagaagtctaacataataagaggctggatttttggtactactt

SEQ ID NO: 37 - RNA, 60 nt, SARS-CoV-2
  source 1..60: mol_type = other RNA
tctgtgaatttcaattttgtaatgatccatttttgggtgtttattaccacaaaaacaaca

SEQ ID NO: 38 - RNA, 60 nt, SARS-CoV-2
  source 1..60: mol_type = other RNA
gccttttcttatggaccttgaaggaaaacagggtaatttcaaaaatcttagggaatttgt

SEQ ID NO: 39 - RNA, 60 nt, SARS-CoV-2
  source 1..60: mol_type = other RNA
gcgtgatctccctcagggtttttcggctttagaaccattggtagatttgccaataggtat

SEQ ID NO: 40 - RNA, 60 nt, SARS-CoV-2
  source 1..60: mol_type = other RNA
ggtgattcttcttcaggttggacagctggtgctgcagcttattatgtgggttatcttcaa

SEQ ID NO: 41 - RNA, 60 nt, SARS-CoV-2
  source 1..60: mol_type = other RNA
tgaccctctctcagaaacaaagtgtacgttgaaatccttcactgtagaaaaaggaatcta

SEQ ID NO: 42 - RNA, 60 nt, SARS-CoV-2
  source 1..60: mol_type = other RNA
atttcctaatattacaaacttgtgcccttttggtgaagtttttaacgccaccagatttgc

SEQ ID NO: 43 - RNA, 60 nt, SARS-CoV-2
  source 1..60: mol_type = other RNA
gcatcattttccacttttaagtgttatggagtgtctcctactaaattaaatgatctctgc

SEQ ID NO: 44 - RNA, 60 nt, SARS-CoV-2
  source 1..60: mol_type = other RNA
ctggaaagattgctgattataattataaattaccagatgattttacaggctgcgttatag

SEQ ID NO: 45 - RNA, 60 nt, SARS-CoV-2
  source 1..60: mol_type = other RNA
ctgtatagattgtttaggaagtctaatctcaaaccttttgagagagatatttcaactgaa

SEQ ID NO: 46 - RNA, 60 nt, SARS-CoV-2
  source 1..60: mol_type = other RNA
atggtttccaacccactaatggtgttggttaccaaccatacagagtagtagtactttctt

SEQ ID NO: 47 - RNA, 60 nt, SARS-CoV-2
  source 1..60: mol_type = other RNA
aaatgtgtcaatttcaacttcaatggtttaacaggcacaggtgttcttactgagtctaac

SEQ ID NO: 48 - RNA, 60 nt, SARS-CoV-2
  source 1..60: mol_type = other RNA
cgtgatccacagacacttgagattcttgacattacaccatgttcttttggtggtgtcagt

SEQ ID NO: 49 - RNA, 60 nt, SARS-CoV-2
  source 1..60: mol_type = other RNA
tgcacagaagtccctgttgctattcatgcagatcaacttactcctacttggcgtgtttat

SEQ ID NO: 50 - RNA, 60 nt, SARS-CoV-2
  source 1..60: mol_type = other RNA
aacaactcatatgagtgtgacatacccattggtgcaggtatatgcgctagttatcagact

SEQ ID NO: 51 - RNA, 60 nt, SARS-CoV-2
  source 1..60: mol_type = other RNA
atgtcacttggtgcagaaaattcagttgcttactctaataactctattgccatacccaca

SEQ ID NO: 52 - RNA, 60 nt, SARS-CoV-2
  source 1..60: mol_type = other RNA
gacatcagtagattgtacaatgtacatttgtggtgattcaactgaatgcagcaatctttt

SEQ ID NO: 53 - RNA, 60 nt, SARS-CoV-2
  source 1..60: mol_type = other RNA
gttgaacaagacaaaaacacccaagaagtttttgcacaagtcaaacaaatttacaaaaca

SEQ ID NO: 54 - RNA, 60 nt, SARS-CoV-2
  source 1..60: mol_type = other RNA
ccaagcaagaggtcatttattgaagatctacttttcaacaaagtgacacttgcagatgct

SEQ ID NO: 55 - RNA, 60 nt, SARS-CoV-2
  source 1..60: mol_type = other RNA
gcacaaaagtttaacggccttactgttttgccacctttgctcacagatgaaatgattgct

SEQ ID NO: 56 - RNA, 60 nt, SARS-CoV-2
  source 1..60: mol_type = other RNA
attacaaataccatttgctatgcaaatggcttataggtttaatggtattggagttacaca

SEQ ID NO: 57 - RNA, 60 nt, SARS-CoV-2
  source 1..60: mol_type = other RNA
tcactttcttccacagcaagtgcacttggaaaacttcaagatgtggtcaaccaaaatgca

SEQ ID NO: 58 - RNA, 60 nt, SARS-CoV-2
  source 1..60: mol_type = other RNA
ttaaatgatatcctttcacgtcttgacaaagttgaggctgaagtgcaaattgataggttg

SEQ ID NO: 59 - RNA, 60 nt, SARS-CoV-2
  source 1..60: mol_type = other RNA
tagagctgcagaaatcagagcttctgctaatcttgctgctactaaaatgtcagagtgtgt

SEQ ID NO: 60 - RNA, 60 nt, SARS-CoV-2
  source 1..60: mol_type = other RNA
tcagtcagcacctcatggtgtagtcttcttgcatgtgacttatgtccctgcacaagaaaa

SEQ ID NO: 61 - RNA, 60 nt, SARS-CoV-2
  source 1..60: mol_type = other RNA
tgtctttgtttcaaatggcacacactggtttgtaacacaaaggaatttttatgaaccaca

SEQ ID NO: 62 - RNA, 60 nt, SARS-CoV-2
  source 1..60: mol_type = other RNA
attgtcaacaacacagtttatgatcctttgcaacctgaattagactcattcaaggaggag

SEQ ID NO: 63 - RNA, 60 nt, SARS-CoV-2
  source 1..60: mol_type = other RNA
tgcttcagttgtaaacattcaaaaagaaattgaccgcctcaatgaggttgccaagaattt

SEQ ID NO: 64 - RNA, 60 nt, SARS-CoV-2
  source 1..60: mol_type = other RNA
gagcagtatataaaatggccatggtacatttggctaggttttatagctggcttgattgcc

SEQ ID NO: 65 - RNA, 60 nt, SARS-CoV-2
  source 1..60: mol_type = other RNA
agttgtctcaagggctgttgttcttgtggatcctgctgcaaatttgatgaagacgactct

SEQ ID NO: 66 - RNA, 60 nt, synthetic construct
  source 1..60: mol_type = other RNA
cacgtgtgaaagaattagtgtatgcagggggtaattgagttctggttgtaagattaacac

SEQ ID NO: 67 - RNA, 60 nt, synthetic construct
  source 1..60: mol_type = other RNA
gtcctgaacaagaatggaaagaaaaggttacaatgaaccaaggtacgatatgtacagaga

SEQ ID NO: 68 - RNA, 60 nt, synthetic construct
  source 1..60: mol_type = other RNA
aaataaaacgaaggtgactcttcagattgtattattctccgacctaaaaaccatgatgaa

SEQ ID NO: 69 - RNA, 60 nt, synthetic construct
  source 1..60: mol_type = other RNA
agacacttaaagttaaaacattactaggtaaaaacccacaaataatggtgtttttgttgt

SEQ ID NO: 70 - RNA, 60 nt, synthetic construct
  source 1..60: mol_type = other RNA
acaaattccctaagatttttgaaattaccctgttttccttcaaggtccataagaaaaggc

SEQ ID NO: 71 - RNA, 60 nt, synthetic construct
  source 1..60: mol_type = other RNA
atacctattggcaaatctaccaatggttctaaagccgaaaaaccctgagggagatcacgc

SEQ ID NO: 72 - RNA, 60 nt, synthetic construct
  source 1..60: mol_type = other RNA
ccactaagaagaagtccaacctgtcgaccacgacgtcgaataatacacccaatagaagtt

SEQ ID NO: 73 - RNA, 60 nt, synthetic construct
  source 1..60: mol_type = other RNA
actgggagagagtctttgtttcacatgcaactttaggaagtgacatctttttccttagat

SEQ ID NO: 74 - RNA, 60 nt, synthetic construct
  source 1..60: mol_type = other RNA
taaaggattataatgtttgaacacgggaaaaccacttcaaaaattgcggtggtctaaacg

SEQ ID NO: 75 - RNA, 60 nt, synthetic construct
  source 1..60: mol_type = other RNA
cgtagtaaaaggtgaaaattcacaatacctcacagaggatgatttaatttactagagacg

SEQ ID NO: 76 - RNA, 60 nt, synthetic construct
  source 1..60: mol_type = other RNA
ctataacgcagcctgtaaaatcatctggtaatttataattataatcagcaatctttccag

SEQ ID NO: 77 - RNA, 60 nt, synthetic construct
  source 1..60: mol_type = other RNA
gacatatctaacaaatccttcagattagagtttggaaaactctctctataaagttgactt

SEQ ID NO: 78 - RNA, 60 nt, synthetic construct
  source 1..60: mol_type = other RNA
taccaaaggttgggtgattaccacaaccaatggttggtatgtctcatcatcatgaaagaa

SEQ ID NO: 79 - RNA, 60 nt, synthetic construct
  source 1..60: mol_type = other RNA
gttagactcagtaagaacacctgtgcctgttaaaccattgaagttgaaattgacacattt

SEQ ID NO: 80 - RNA, 60 nt, synthetic construct
  source 1..60: mol_type = other RNA
gcactaggtgtctgtgaactctaagaactgtaatgtggtacaagaaaaccaccacagtca

SEQ ID NO: 81 - RNA, 60 nt, synthetic construct
  source 1..60: mol_type = other RNA
ataaacacgccaagtaggagtaagttgatctgcatgaatagcaacagggacttctgtgca

SEQ ID NO: 82 - RNA, 60 nt, synthetic construct
  source 1..60: mol_type = other RNA
ttgttgagtatactcacactgtatgggtaaccacgtccatatacgcgatcaatagtctga

SEQ ID NO: 83 - RNA, 60 nt, synthetic construct
  source 1..60: mol_type = other RNA
tgtgggtatggcaatagagttattagagtaagcaactgaattttctgcaccaagtgacat

SEQ ID NO: 84 - RNA, 60 nt, synthetic construct
  source 1..60: mol_type = other RNA
ctgtagtcatctaacatgttacatgtaaacaccactaagttgacttacgtcgttagaaaa

SEQ ID NO: 85 - RNA, 60 nt, synthetic construct
  source 1..60: mol_type = other RNA
caacttgttctgtttttgtgggttcttcaaaaacgtgttcagtttgtttaaatgttttgt

SEQ ID NO: 86 - RNA, 60 nt, synthetic construct
  source 1..60: mol_type = other RNA
ggttcgttctccagtaaataacttctagatgaaaagttgtttcactgtgaacgtctacga

SEQ ID NO: 87 - RNA, 60 nt, synthetic construct
  source 1..60: mol_type = other RNA
cgtgttttcaaattgccggaatgacaaaacggtggaaacgagtgtctactttactaacga

SEQ ID NO: 88 - RNA, 60 nt, synthetic construct
  source 1..60: mol_type = other RNA
taatgtttatggtaaacgatacgtttaccgaatatccaaattaccataacctcaatgtgt

SEQ ID NO: 89 - RNA, 60 nt, synthetic construct
  source 1..60: mol_type = other RNA
agtgaaagaaggtgtcgttcacgtgaaccttttgaagttctacaccagttggttttacgt

SEQ ID NO: 90 - RNA, 60 nt, synthetic construct
  source 1..60: mol_type = other RNA
caacctatcaatttgcacttcagcctcaactttgtcaagacgtgaaaggatatcatttaa

SEQ ID NO: 91 - RNA, 60 nt, synthetic construct
  source 1..60: mol_type = other RNA
atctcgacgtctttagtctcgaagacgattagaacgacgatgattttacagtctcacaca

SEQ ID NO: 92 - RNA, 60 nt, synthetic construct
  source 1..60: mol_type = other RNA
agtcagtcgtggagtaccacatcagaagaacgtacactgaatacagggacgtgttctttt

SEQ ID NO: 93 - RNA, 60 nt, synthetic construct
  source 1..60: mol_type = other RNA
acagaaacaaagtttaccgtgtgtgaccaaacattgtgtttccttaaaaatacttggtgt

SEQ ID NO: 94 - RNA, 60 nt, synthetic construct
  source 1..60: mol_type = other RNA
taacagttgttgtgtcaaatactaggaaacgttggacttaatctgagtaagttcctcctc

SEQ ID NO: 95 - RNA, 60 nt, synthetic construct
  source 1..60: mol_type = other RNA
acgaagtcaacatttgtaagtttttctttaactggcggagttactccaacggttcttaaa

SEQ ID NO: 96 - RNA, 60 nt, synthetic construct
  source 1..60: mol_type = other RNA
ctcgtcatatattttaccggtaccatgtaaaccgatccaaaatatcgaccgaactaacgg

SEQ ID NO: 97 - RNA, 60 nt, synthetic construct
  source 1..60: mol_type = other RNA
agagtcgtcttcatcaaatttgcagcaggatccacaagaacaacagcccttgagacaact

SEQ ID NO: 98 - RNA, 29 nt, SARS-CoV-2
  source 1..29: mol_type = other RNA
tgtttttcttgttttattgccactagtct

SEQ ID NO: 99 - RNA, 29 nt, SARS-CoV-2
  source 1..29: mol_type = other RNA
accctgacaaagttttcagatcctcagtt

SEQ ID NO: 100 - RNA, 29 nt, SARS-CoV-2
  source 1..29: mol_type = other RNA
ggtactaagaggtttgataaccctgtcct

SEQ ID NO: 101 - RNA, 29 nt, SARS-CoV-2
  source 1..29: mol_type = other RNA
cccagtccctacttattgttaataacgct

SEQ ID NO: 102 - RNA, 29 nt, SARS-CoV-2
  source 1..29: mol_type = other RNA
gtgagttcagagtttattctagtgcgaat

SEQ ID NO: 103 - RNA, 29 nt, SARS-CoV-2
  source 1..29: mol_type = other RNA
tattctaagcacacgcctattaatttagt

SEQ ID NO: 104 - RNA, 29 nt, SARS-CoV-2
  source 1..29: mol_type = other RNA
catcactaggtttcaaactttacttgctt

SEQ ID NO: 105 - RNA, 29 nt, SARS-CoV-2
  source 1..29: mol_type = other RNA
ggaaccattacagatgctgtagactgtgc

SEQ ID NO: 106 - RNA, 29 nt, SARS-CoV-2
  source 1..29: mol_type = other RNA
acttctaactttagagtccaaccaacaga

SEQ ID NO: 107 - RNA, 29 nt, SARS-CoV-2
  source 1..29: mol_type = other RNA
gcaactgtgttgctgattattctgtccta

SEQ ID NO: 108 - RNA, 29 nt, SARS-CoV-2
  source 1..29: mol_type = other RNA
gaggtgatgaagtcagacaaatcgctcca

SEQ ID NO: 109 - RNA, 29 nt, SARS-CoV-2
  source 1..29: mol_type = other RNA
ctaacaatcttgattctaaggttggtggt

SEQ ID NO: 110 - RNA, 29 nt, SARS-CoV-2
  source 1..29: mol_type = other RNA
cttgtaatggtgttgaaggttttaattgt

SEQ ID NO: 111 - RNA, 29 nt, SARS-CoV-2
  source 1..29: mol_type = other RNA
agcaactgtttgtggacctaaaaagtcta

SEQ ID NO: 112 - RNA, 29 nt, SARS-CoV-2
  source 1..29: mol_type = other RNA
ccaacaatttggcagagacattgctgaca

SEQ ID NO: 113 - RNA, 29 nt, SARS-CoV-2
  source 1..29: mol_type = other RNA
gaacaaatacttctaaccaggttgctgtt

SEQ ID NO: 114 - RNA, 29 nt, SARS-CoV-2
  source 1..29: mol_type = other RNA
taatgtttttcaaacacgtgcaggctgtt

SEQ ID NO: 115 - RNA, 29 nt, SARS-CoV-2
  source 1..29: mol_type = other RNA
cgggcacgtagtgtagctagtcaatccat

SEQ ID NO: 116 - RNA, 29 nt, SARS-CoV-2
  source 1..29: mol_type = other RNA
agtgttaccacagaaattctaccagtgtc

SEQ ID NO: 117 - RNA, 29 nt, SARS-CoV-2
  source 1..29: mol_type = other RNA
ggcagtttttgtacacaattaaaccgtgc

SEQ ID NO: 118 - RNA, 29 nt, SARS-CoV-2
  source 1..29: mol_type = other RNA
ggtggttttaatttttcacaaatattacc

SEQ ID NO: 119 - RNA, 29 nt, SARS-CoV-2
  source 1..29: mol_type = other RNA
aatatggtgattgccttggtgatattgct

SEQ ID NO: 120 - RNA, 29 nt, SARS-CoV-2
  source 1..29: mol_type = other RNA
ttagcgggtacaatcacttctggttggac

SEQ ID NO: 121 - RNA, 29 nt, SARS-CoV-2
  source 1..29: mol_type = other RNA
attgattgccaaccaatttaatagtgcta

SEQ ID NO: 122 - RNA, 29 nt, SARS-CoV-2
  source 1..29: mol_type = other RNA
ttgttaaacaacttagctccaattttggt

SEQ ID NO: 123 - RNA, 29 nt, SARS-CoV-2
  source 1..29: mol_type = other RNA
agacttcaaagtttgcagacatatgtgac

SEQ ID NO: 124 - RNA, 29 nt, SARS-CoV-2
  source 1..29: mol_type = other RNA
caatcaaaaagagttgatttttgtggaaa

SEQ ID NO: 125 - RNA, 29 nt, SARS-CoV-2
  source 1..29: mol_type = other RNA
ccatttgtcatgatggaaaagcacacttt

SEQ ID NO: 126 - RNA, 29 nt, SARS-CoV-2
  source 1..29: mol_type = other RNA
acaacacatttgtgtctggtaactgtgat

SEQ ID NO: 127 - RNA, 29 nt, SARS-CoV-2
  source 1..29: mol_type = other RNA
ttaagaatcatacatcaccagatgttgat

SEQ ID NO: 128 - RNA, 29 nt, SARS-CoV-2
  source 1..29: mol_type = other RNA
aatctctcatcgatctccaagaacttgga

SEQ ID NO: 129 - RNA, 29 nt, SARS-CoV-2
  source 1..29: mol_type = other RNA
ggtgacaattatgctttgctgtatgacca

SEQ ID NO: 130 - RNA, 29 nt, SARS-CoV-2
  source 1..29: mol_type = other RNA
tcaaaggagtcaaattacattacacataa